(19) United States
(12) Patent Application Publication
(10) Pub. No.: US 2026/0298902 A1
(43) Pub. Date: Oct. 1, 2026

(54) APPARATUS AND SYSTEM FOR IDENTIFYING PRESENCE OF A SUBSTANCE OF INTEREST, AND METHOD OF USING THEREOF

(71) Applicant: Get Wisp Ltd., Givat Haim Meuhad (IL)

(72) Inventors: David AVRAHAM, Givat Haim Meuhad (IL); Aviv KAPLAN, Givat Haim Meuhad (IL)

(21) Appl. No.: 19/478,138

(22) PCT Filed: Apr. 24, 2024
(86) PCT No.: PCT/IB2024/054000

§ 371 Date: Oct. 23, 2025

(30) Foreign Application Priority Data
Apr. 25, 2023 (IL) 302443

(51) Int. Cl. G01N 33/14; G01N 21/47

(52) U.S. Cl. CPC G01N 33/146; G01N 21/47; G01N 2021/4709

(57) ABSTRACT

An apparatus for detecting a chemical substance may include a radiation source configured to produce radiation of a first wavelength and an optical system configured to (i) illuminate a target object in a first direction by the radiation, (ii) receive back-scattered radiation from the first direction, in one or more second wavelengths, and (iii) direct the back-scattered radiation to a detector device, configured to measure intensity of the back-scattered radiation in at least one of the second wavelengths. The optical system may include an optical path that defines a plane that is substantially orthogonal to the first direction. The apparatus may also include at least one processor, configured to identify presence of a chemical substance of interest at the target object, based on the measured intensity.

### CROSS-REFERENCE TO RELATED APPLICATIONS

This application is a 371 of international patent application PCT/IB2024/054000, which claims priority and/or benefit from IL application 302443 filed Apr. 25, 2023, titled “APPARATUS FOR IDENTIFYING PRESENCE OF SUBSTANCE OF INTEREST, AND METHOD OF USING THEREOF”, and which are incorporated herein by reference in their entirety.

### TECHNICAL FIELD

The present invention relates generally to the technological field of remote detection. More specifically, the present invention relates to an apparatus for using spectroscopic technology for identifying the presence of a substance of interest.

### BACKGROUND

The utilization of Raman spectroscopy for identifying presence of substances of interest is ubiquitous, and facilitates a wide variety of applications. However, currently available techniques and/or methods typically involves expensive, dedicated hardware modules.

Currently available Raman spectroscopy applications which make use of cost-effective, available smartphone-based spectrometers, require two separate modules, i.e., a transmitter and a receiver. These modules are typically positioned in an arbitrary (e.g., 90 degree) geometry in relation to an examined object, to receive, and analyze reflectance of radiation from the examined object.

This configuration has several disadvantages, which contribute to inefficiency, inaccuracy and unpredictability of currently available systems. For example, currently available smartphone-based spectrometers require maintenance, calibration, and accurate, repeatable positioning of the different modules. Additionally, such smartphone-based spectrometers cannot employ the smartphone to efficiently control, and/or or obtain feedback for the process of spectrometric measurement.

An apparatus, system and method of performing Raman spectroscopy measurements in a cost-effective and repeatable manner, for reliably identifying a substance in an object of interest is therefore required.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for identifying presence of a substance of interest, according to some embodiments.

FIG. 2 is a block diagram, depicting a system and apparatus identifying presence of a substance of interest, according to some embodiments.

FIG. 3A is a schematic diagram depicting an application of currently available systems for performing Raman spectroscopy measurements, according to some embodiments.

FIG. 3B is a schematic diagram depicting employment of an apparatus for performing Raman spectroscopy measurements, according to some embodiments.

FIGS. 4A-4D are schematic diagrams depicting different configurations of an optical system of an apparatus for identifying presence of a substance of interest, according to some embodiments.

FIGS. 5A and 5B are schematic diagrams depicting configurations of a separator component, which may be included in an apparatus for identifying presence of a substance of interest, according to some embodiments.

FIGS. 6A and 6B are isometric views depicting an example of implementation of a separator component, which may include a pair of dichroic mirrors, and may be included in an apparatus for identifying presence of a substance of interest, according to some embodiments.

FIGS. 6C and 6D are isometric views depicting another example of implementation of a separator component, which may include a pair of mirrors, and may be included in an apparatus for identifying presence of a substance of interest, according to some embodiments of the invention. At least one of the pair of mirrors may be a dichroic mirror, and at least one of the pair of mirrors may have a concave geometry, according to some embodiments.

FIGS. 6E1 and 6E2 are diagrams of an example of a separator component, which may be included in an apparatus for identifying presence of a substance of interest, according to some embodiments.

FIG. 7 is a flow diagram, depicting a method of identifying presence of a substance of interest in a target object, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, and/or other electronic computing device, that manipulates and/or transforms data represented as physical (e.g., electronic) quantities within the computer's registers and/or memories into other data similarly represented as physical quantities within the computer's registers and/or memories and/or other information in a transitory and/or non-transitory storage medium that may store instructions to perform operations and/or processes. A transitory storage medium may include a propagating signal.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention may include an apparatus, and a system for investigating a target object, for example, for detecting an anomaly relating to the target object such as, for example, identifying presence of a chemical substance of interest in a target object, and a method of using thereof.

The system is configured to output (e.g., display) to the user information about a detected anomaly and/or a measured quantity and/or probability with respect to the presence of an anomaly (e.g., expected and/or detected quantity of a certain target analyte). The system may also be configured to output (e.g., display) information with respect to a non-finding of an anomaly.

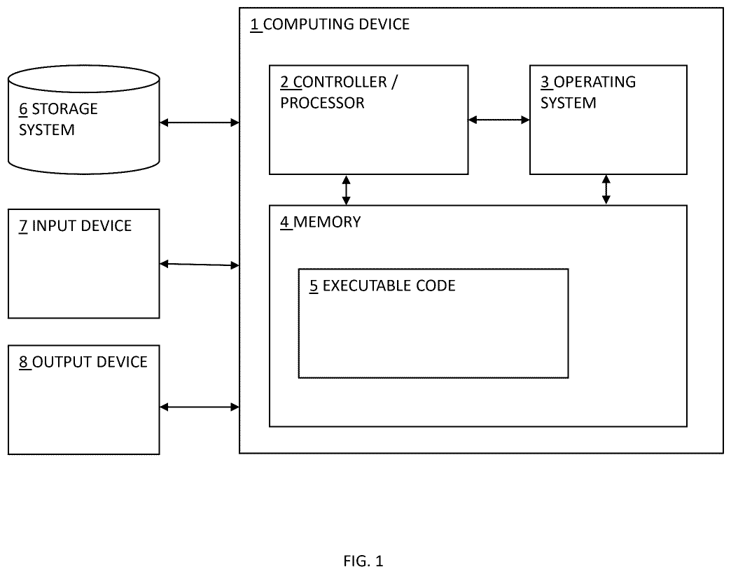

Reference is now made to FIG. 1, which is a block diagram depicting a computing device, which may be included within an embodiment of a system and apparatus for identifying presence of a chemical substance of interest in a target object (e.g., gaseous and/or liquid fluid), according to some embodiments of the invention.

Computing device 1 may include a processor and/or controller 2 that may be, for example, a central processing unit (CPU) processor, a chip and/or any suitable computing and/or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute and/or act as the various modules, units, etc. More than one computing device 1 may be included in, and one or more computing devices 1 may act as the components of, a system according to embodiments of the invention.

Operating system 3 may be and/or may include any code segment (e.g., one similar to executable code 5 described herein) designed and/or configured to perform tasks involving coordination, scheduling, arbitration, supervising, controlling and/or otherwise managing operation of computing device 1, for example, scheduling execution of software programs and/or tasks and/or enabling software programs and/or other modules and/or units to communicate. Operating system 3 may be a commercial operating system. It will be noted that an operating system 3 may be an optional component, e.g., in some embodiments, a system may include a computing device that does not require and/or include an operating system 3.

Memory 4 may be and/or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, and/or other suitable memory units and/or storage units. Memory 4 may be and/or may include a plurality of possibly different memory units. Memory 4 may be a computer and/or processor non-transitory readable medium, and/or a computer non-transitory storage medium, e.g., a RAM. In one embodiment, a non-transitory storage medium such as memory 4, a hard disk drive, another storage device, etc. may store instructions and/or code which when executed by a processor may cause the processor to carry out methods as described herein.

Executable code 5 may be any executable code, e.g., an application, a program, a process, task, and/or script. Executable code 5 may be executed by processor and/or controller 2 possibly under control of operating system 3. For example, executable code 5 may be an application that may identify the presence of one or more chemical substances of interest in a target object as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be and/or may include, for example, a flash memory, e.g., as known in the art, a memory that is internal to, and/or embedded in, a micro controller and/or chip, e.g., as known in the art, a hard disk drive, a CD-Recordable (CD-R) drive, a Blu-ray disk (BD), a universal serial bus (USB) device and/or other suitable removable and/or fixed storage unit. Data pertaining to a spectrum of radiation, reflected from a target object of interest (e.g., a surface of liquid material) may be stored in storage system 6 and may be loaded from storage system 6 into memory 4 where it may be processed by processor and/or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded and/or included in memory 4.

Input devices 7 may be and/or may include any suitable input devices, components, and/or systems, e.g., a detachable keyboard and/or keypad, a mouse, and/or the like. Output devices 8 may include one or more (possibly detachable) displays and/or monitors, speakers and/or any other suitable output devices. Any applicable input/output (I/O) devices may be connected to Computing device 1 as shown by blocks 7 and 8. For example, a wired and/or wireless network interface card (NIC), a universal serial bus (USB) device and/or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) and/or any other suitable multi-purpose and/or specific processors and/or controllers (e.g., similar to element 2), a plurality of input units, a plurality of output units, a plurality of memory units, and a plurality of storage units.

The term neural network (NN) and/or artificial neural network (ANN), e.g., a neural network implementing a machine learning (ML) and/or artificial intelligence (AI) function, may be used herein to refer to an information processing paradigm that may include nodes, referred to as neurons, organized into layers, with links between the neurons. The links may transfer signals between neurons and may be associated with weights. A NN may be configured and/or trained for a specific task, e.g., pattern recognition and/or classification. Training a NN for the specific task may involve adjusting these weights based on examples. Each neuron of an intermediate and/or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one and/or more CPUs and/or graphics processing units (GPUs), and/or a dedicated hardware device may perform the relevant calculations.

As used herein the term “machine learning” refers to a procedure embodied as a computer program configured to induce patterns, regularities, and/or rules from previously collected data to develop an appropriate response to future data and/or describe the data in some meaningful way.

Examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

The machine learning procedure used according to some embodiments of the present invention is a trained machine learning procedure, which provides output that is related non-linearly to the parameters with which it is fed.

In some embodiments, a machine learning procedure can be trained according to some embodiments by feeding a machine learning training program with parameter values that characterize various target objects (e.g., beverages) that have been identified as containing or not containing an undesired substance.

Once the data is fed, the machine learning training program generates a trained machine learning procedure and/or forms a part of a ML module. In some examples, the trained ML module can be used without the need to re-train it. In some other examples, the trained ML module may be further trained and tested, e.g., on-the-fly, by training the ML module with data relating to a target object currently analyzed. In some embodiments, the ML model may trained with additional labels including, for example, beverage metainformation; with labels relating to physiological and/or socio-economic and/or behavioral user characteristics such as, for example, gender, age, race, height, BMI, smoking habits, drinking habits, medical history, and/or the like. For example, the fact that the user is female may be associated with an increased probability that a beverage about to be consumed by the same user contains an undesired substance.

An ML model that may be employed by the system may be trained using supervised and/or semi-supervised and/or unsupervised learning. In some examples, the machine learning model may be adapted by evaluating labels produced by a test dataset. The validation measures may include, for example, accuracy, recall and/or precision, with respect to real labels on a dataset of labeled data.

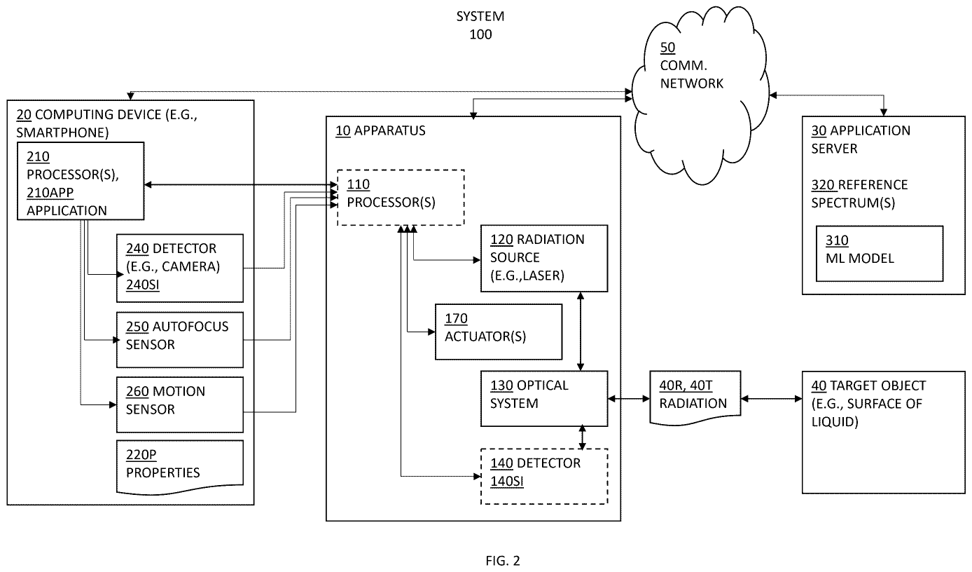

Reference is now made to FIG. 2, which depicts a system 100 for identifying presence of a chemical substance of interest in a target object, according to some embodiments of the invention.

According to some embodiments of the invention, system 100 may be implemented as a software module, a hardware module, and/or any combination thereof. For example, system may be and/or may include a computing device such as element 1 of FIG. 1, and may be adapted to execute one or more modules of executable code (e.g., element 5 of FIG. 1) to identify presence of a chemical substance of interest in a target object 40, as further described herein.

As shown in FIG. 2, arrows may represent flow of one or more data elements to and from system 100 and/or among modules or elements of system 100. Some arrows have been omitted in FIG. 2 for the purpose of clarity.

System 100 may include an apparatus 10, configured to obtain spectrometric data characterizing a target object 40. According to some embodiments, apparatus 10 may include one or more processors 110 configured to analyze the spectrometric data, to determine presence of a material of interest at, or in the target object 40.

In an exemplary application, target object 40 may be a body of liquid, such as a glass of beverage or drink. Apparatus 10 may then be employed to determine whether an unwanted substance, such as a drug, has been added to that drink.

Additionally, or alternatively, and as shown in the implementation example of FIG. 2, apparatus 10 may collaborate with at least one other processor to perform this analysis.

For example, apparatus 10 may be attached to, or mounted on a computing device 20 such as a smartphone. Apparatus 10 may collaborate with a processor 210 of a computing device (e.g., smartphone) 20, to analyze the spectrometric data, and determine presence of the material of interest at the target object 40, as elaborated herein.

The term computing device 20 may be used herein in a generic form, to indicate a communication computing device that may include an integrated detector device such as, for example, a camera. The computing device may, in some examples, be substantially flat. For example, computing device 20 may be a smartphone, a “tablet” computing device, and/or the like. In this context, the terms “computing device” 20 and “smartphone” 20 may be used interchangeably.

Additionally, or alternatively, apparatus 10 and/or computing device 20 may be associated with, or communicatively connected, via a communication network 50 (e.g., cellular network, the Internet, etc.) to at least one server computing device 30 (e.g., computing device 1 of FIG. 1), also referred to herein as application server 30. In such embodiments, apparatus 10 and server computing device 30 may include a model (e.g., classifier, analysis engine) such as, for example, a machine learning (ML)-based model 310.

Although embodiments disclosed herein may pertain to a ML model, this should by no means be construed in a limiting manner. In some examples, an analysis engine may implement and/or comprise in addition and/or as an alternative to a (trained) machine learning model, a rule-based machine, or a combined ML and rule-based model.

In some examples, the analysis engine may be implemented by a computing device (e.g., Smartphone), a server, or both.

In some embodiments, a machine learning model may be trained based on data relating to one or more properties and/or characteristics (e.g., fluid data elements) relating to a target sample (e.g., a liquid such as, for example, a drink) including, for example, spectral data representative of a target, spectral data representative of a target sample containing or comprising a chemical substance of interest (also: chemical target analyte); an image of the target substance, a target sample type; a target brand; a place of purchase of the target sample, and/or the like. The terms “target object” and “target sample”, may herein be used interchangeably.

In some embodiments, a trained ML model may receive data relating to a target object and determine, based on the received data, an output relating to a substance of interest contained in the target object.

In some embodiments, the trained ML model may be configured to detect an anomaly relating to the target object. For example, the trained ML model may be configured to detect, based on data relating to the target object (e.g., data relating to spectral information, purchase location, brand, type of drink, shape of receptacle, etc.), an anomaly relating to the scanned target object. In some examples, a type of drink may relate to a family of drinks (e.g., spirits, cocktails, distilled alcoholic beverages, undistilled alcoholic beverages), and/or to a type of drink within a certain family (e.g., type of wine, type of beer, type of spirits, etc.). For example, certain metadata (e.g., certain brands, purchase locations, types of drinks, purchase times, and/or the like), may be given higher weighting with respect to the probability of the presence of an unwanted drug, compared to other beverage metadata.

The trained ML model may be configured to analyze data (also: drink property data elements) relating to different information of the target object, by taking into consideration the collective contribution of the data descriptive of different information relating to the target object for identifying an anomaly relating to the target object.

In some embodiments, an ML model may be trained based on measurements taken with respect to a reference target object containing a known level of a desired substance (e.g., 10% alcohol level). In some embodiments, based on spectral output of a single scan (e.g., reference scan), artificial spectral scan data may be artificially generated, which may be employed as training data. In some examples, the artificial spectral scan data may be generated using artificially and/or forcefully altered scanning conditions (e.g., different equipment operating parameter values, different ambient conditions, and/or the like, resulting in varying outputs), for artificially generating multiple labelled data for reference, which may be utilized for training the ML model. In some embodiments, the obtained outputs may vary with respect to SNR, resolution, laser performance, sensor performance, and/or the like.

In some embodiments, the (e.g., trained ML) model may be configured to distinguish between two or more anomalies that relate to a desired substance in the target sample such as, for example, Ethanol concentration, for example, to detect whether the beverage and/or the receptacle and/or label has been illicitly tampered (e.g., diluted with water, re-labeled to conceal true origin, addition of toxic substances). For example, the model may be configured to distinguish between two or more anomalies that relate to an unwanted substance in the target sample such as, for example, content of incapacitating agents (first anomaly) and methanol (second anomaly) present in an alcoholic beverage.

In some embodiments, the model may be configured to distinguish between toxic and non-toxic dosage anomalies of the target object. For example, the model may be configured to determine whether a toxic substance (e.g., incapacitating agent) has been added to the target object, and/or whether a non-toxic substance (water) yet still unwanted substance has been added to the target object, e.g., to dilute the ethanol level in the beverage.

In some embodiments, in case an anomaly is detected, the system may output whether the anomaly relates to a toxic or a non-toxic anomaly. In some embodiments, the system may different between different types of toxic anomalies, e.g., whether an incapacitating agent has been added to the beverage, and/or whether the beverage was illicitly tampered with Methanol, for example, and/or any other drug that does not alter a person's state of consciousness.

It is noted that term “toxic” refers to a dose in the target object that may cause a detectable toxic effect.

It is noted that where applicable, the terms “system”, and “model” may herein be used interchangeably. In some examples, a system and/or an apparatus may comprise an (ML) model.

Unless otherwise stated, the term “alcohol” as used herein pertains to Ethanol contained at non-toxic levels in an alcoholic beverage.

An anomaly relating to the investigated target object may be detected, for example, if a measured parameter value (e.g., spectral parameter value or range) exceeds above or drops below a certain threshold. The measured parameter value may pertain to a desired and/or expected analyte quantity or level (e.g., concentration) in the target object (e.g., alcohol concentration in a beverage), an undesired analyte in the target object (e.g., rape and/or other drug substance concentration in the beverage), and/or the like. It is noted that the term “quantity” as used herein may also encompass “quantity range”. The terms “quantity”, “quantity measure”, “level”, and/or “measure”, may herein be used interchangeably and may, for example, pertain to a relative quantity (e.g., analyte concentration), to an absolute quantity, and/or a probability.

In some embodiments, the system may be configured to determine a measure (e.g., probability) relating to the presence of an unwanted analyte in the target object.

In some embodiments, an anomaly may be detected if the determined measure exceeds a certain threshold or drops below a certain threshold.

In some embodiments, a measured quantity of a first (e.g., desired) analyte in the target object may be used for determining a probability of the presence of a second (e.g., undesired) analyte in the same target object, and/or vice versa, for example, through transfer learning. In some examples, based on a model employed for determining the presence of a first target analyte, the presence of a second target analyte may be determined, and/or vice versa.

In some embodiments, the system may test a beverage for either a desired and undesired target analyte and determine, based on the outcome, whether further testing is required, e.g., to reduce the probability of false-positive and/or false-negative results with respect to the presence of the undesired target analyte. If a discrepancy between the measured and/or obtained information exceeds a threshold, the system may initiate further tests. In some embodiments, the system may prompt the user to perform additional tests. In some embodiments, an additional test or additional set of tests, for the same target object, may be conducted. The additional test(s) may be performed using equal or different operating parameter values than those under which an initial test or initial set of tests was performed.

In some embodiments, operating parameters may pertain to equipment operating parameters (e.g., laser operating parameters, camera operating parameters, etc.), environmental parameters, and/or the like.

It is noted that the term “determining” may also encompass the meaning of the term “determining an estimate”.

In some embodiments, a (e.g., regression) model (or any other classification model) may be trained through transfer learning. For example, a first model employed for drug detection may be employed for deriving a second model for alcohol detection. In some examples, a model employed for alcohol detection may be employed for deriving a model for drug detection. In some examples, alcohol detection may be based on drug detection model, and/or vice versa.

For instance, the model (also: analysis engine) may be “transfer-learning trained”, e.g., as described herein.

In some embodiments, based on a measured quantity of a desired analyte agent (e.g., ethanol), an expected quantity and/or probability of an undesired target analyte in the same beverage (e.g., incapacitating agent) may be determined.

In some embodiments, a measured quantity of a desired analyte (e.g., alcohol concentration in a beverage) may be compared against an expected quantity of the desired analyte. If the measured quantity of the desired analyte drops below a threshold, an increase in the probability that an undesired is present in the same target object may be determined. In some examples, if a measured quantity of a desired analyte about equals an expected quantity, a reduced probability regarding the presence of the undesired analyte may be determined. If a measured quantity of a desired analyte exceeds an expected quantity, it may be determined that the beverage has been tampered with regarding the desired analyte (e.g., alcohol is added to the beverage). Optionally, a reduced probability regarding the presence of the undesired analyte may then be determined.

In some embodiments, if despite an unexpected change in a measured quantity of a desired analyte (e.g., alcohol drop below a threshold), the undesired analyte remains undetected or below an expected measured quantity, the system may trigger a protocol relating to a potential false-negative identification with respect to the undesired analyte. In such a case, the system may request that the user performs repeated testing (e.g., repeated beverage scanning, providing additional information about the beverage, and/or the like), to reduce the probability or rule out false-negative identification with respect to presence of an undesired analyte in the target object.

In some examples, in case the value of the desired analyte remains below an expected threshold, yet no undesired analyte concentration is detected, the system may output a warning, indicating that the beverage may contain an undesired analyte not accounted for by the system, e.g., because the drug is not yet known, data descriptive of the drug characteristics are not available for comparison in the system, and/or due to malfunction of a system component.

In some examples, information relating to an expected or desired analyte (e.g., an expected alcohol concentration in a beverage) may be derived by the system, for example, through spectrometry, received by the system (e.g., through image acquisition of a label associated with (e.g., attached to) a receptacle containing the beverage, and based on analysis of the image of the label; based on information provided by a user of the system, and/or the like). The expected alcohol concentration may be compared against an actual alcohol concentration measured by the system (e.g., by applying spectrometry). If the actual alcohol concentration is below an expected alcohol concentration, it may be derived that the beverage has been tampered with, e.g., through the addition of an unwanted substance, resulting in a drop in the alcohol concentration.

In some embodiments, based on a measured quantity of an undesired analyte agent (e.g., incapacitating agent concentration in a beverage), an expected or predicted quantity of the desired analyte may be determined.

In some examples, the expected quantity of the desired target analyte, may be compared against metainformation available for the same beverage, obtained, e.g., by through image analysis of a label attached to the user's receptacle that contains the beverage or from which the beverage was poured into the user's receptacle.

In some examples, if the presence of undesired analyte is detected, a reduction in the quantity of the desired target agent is expected to be measured.

In some examples, if there is a discrepancy between a measured or expected desired target analyte quantity, and obtained metainformation about the desired target analyte (e.g., the measured quantity of desired target analyte is below an expected quantity of the same desired target analyte), the system may issue an output indicative of the increased probability of the presence of a undesired target agent in the beverage, e.g., even if there is no measured quantity in the undesired target analyte. In such a scenario, the user may be prompted or asked to conduct additional tests. This way, the probability of false-negative results may be reduced.

In some examples, if there is no discrepancy between a measured or expected undesired target analyte quantity and obtained metainformation about a desired target analyte in the same target analyte (e.g., a measured quantity of the undesired target analyte is detected, yet there is no drop in the measured quantity of the desired quantity), the system may issue an output indicative of the increased probability of the presence of an undesired target agent in the beverage. In such a scenario, the user may optionally be requested to perform additional tests. This way, the probability of false-positive errors may be reduced.

In some embodiments, the system may comprise one or more environmental sensors configured sense a physical quantity relating to the environment the target object (e.g., humidity, ambient temperature, ambient pressure). In some embodiments, the system may comprise one or more fluid sensors configured to sense non-spectral quantities relating to the target object (e.g., fluid temperature). In some embodiments, the system may comprise one or more system monitoring sensors configured to monitor system hardware components. Outputs of any one of the one more sensors may be taking into consideration by the system when performing spectrometry and/or processing acquired spectral data for determining a probability relating to the presence of one or more anomalies with respect to the target object, e.g., to reduce false-positives and/or false-negative outputs.

In some embodiments, the term “receptacle” may pertain to a previously sealed receptacle containing a beverage (e.g., beer can). In some embodiments, the term “receptacle” may pertain to a receptacle into which the contents of a previously sealed receptacle are poured into (e.g., a wine glass, a beer glass).

In some examples, server 30 may be configured to accumulate data from one or more apparatuses 10, to train the (e.g., ML) model to arrive at the trained ML model, for example, to account for differences in performance of different apparatuses 10.

In some examples, a trained ML model may be configured to determine or predict expected spectral data representative of scanned objects (e.g., a plurality of drinks, of different types and brands) and/or substances of interest (e.g., a plurality of unwanted substances), as elaborated herein. In a subsequent inference stage, apparatus 10 and/or smartphone device 20 may transmit measured spectrometric data (140SI and/or 240SI respectively), characterizing a target object 40 (e.g., a new, specific glass of drink) to application server 30, which may infer a trained ML model (e.g., model 310) on this data, to identify presence of a substance of interest (e.g., a suspected drug) in target object 40. A type of object may pertain to a type of beverage including, for example, alcoholic beverages, non-alcoholic beverages. In some examples, a type may pertain to a type of alcoholic beverage (e.g., beer, wine, spirits); a type of non-alcoholic beverage (e.g., juice, carbonated soft drink); and/or any combination of the aforesaid such as, for example, a cocktail (e.g., alcoholic mixed drink or non-alcoholic mixed drink). A type of drink may be associated with an expected alcohol concentration.

It may be appreciated by a person skilled in the art that other such combinations of distributed analysis of measured spectrometric data 140SI/240SI, containing wavelengths and amplitudes of Raman radiation peaks, by one or more processing units (e.g., processor 2 of FIG. 1) may also be possible.

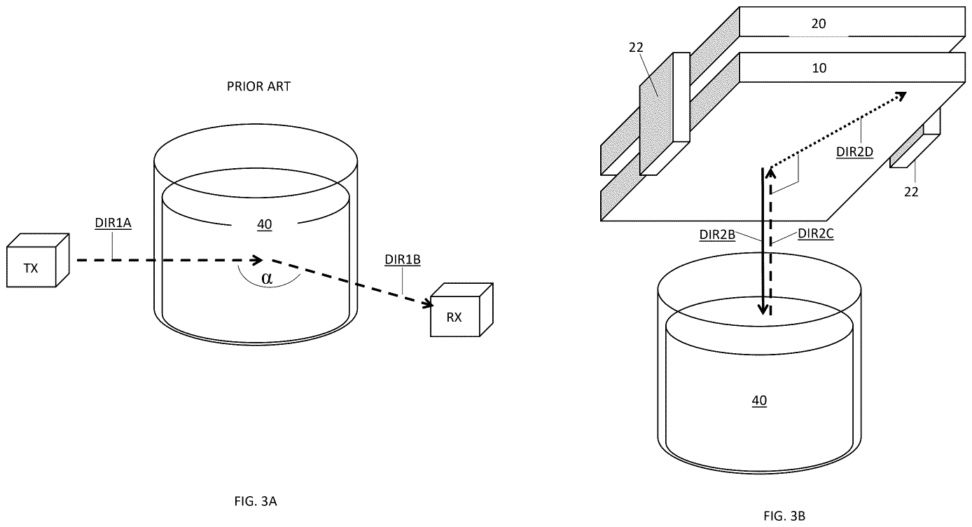

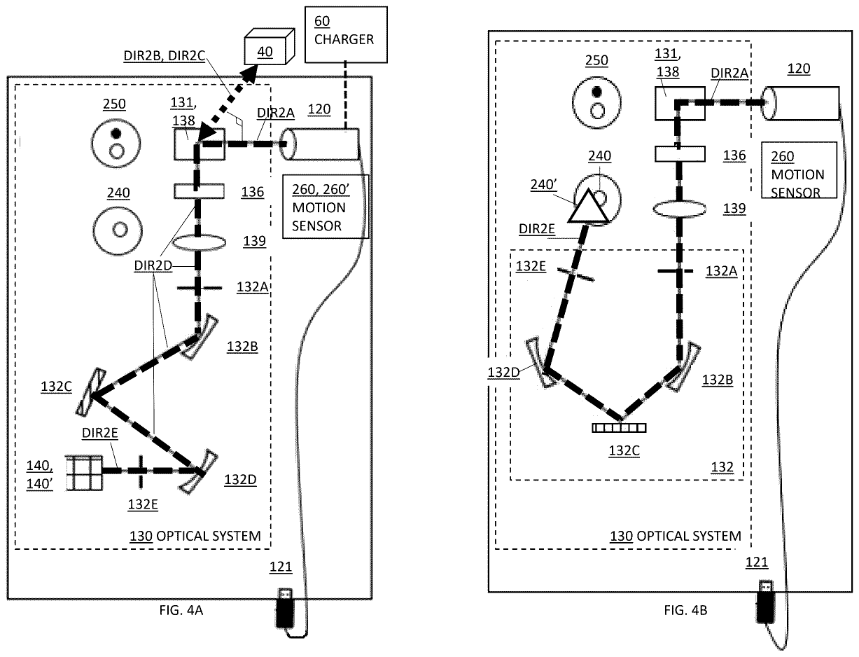

Reference is now made to FIG. 3A, representing application of currently available systems for performing Raman spectroscopy measurements, and to FIG. 3B, showing employment of apparatus 10 of system 100 for performing Raman spectroscopy measurements, according to some embodiments of the invention.

As shown in FIG. 3A, Raman spectroscopy technology application of smartphone-based Raman spectrometers requires two separate modules, i.e., a transmitter (denoted TX) and a receiver (denoted RX). These modules are typically positioned in an arbitrary geometry in relation to an examined object 40. For example, RX, TX and examined object 40 may be positioned in a 90-degrees configuration (denoted by α) between directions DIR1A and DIR1B), to illuminate object 40, and subsequently receive, and analyze reflectance of radiation from examined object 40.

Currently available smartphone-based spectrometers such as, for example, depicted in FIG. 3A present several disadvantages, which contribute to inefficiency, inaccuracy and unpredictability of currently available systems, and may even produce false measurement results.

For example, systems such as in FIG. 3A require acquisition and maintenance of both a transmitting unit and a receiving unit, as well as calibration between these two units, to ensure proper interoperability and compatibility.

In another example, systems such as, for example, in FIG. 3A require accurate, repeatable positioning of the different modules (RX and TX), in relation to the examined object 40.

In another example, systems such as, for example, in FIG. 3A are inconvenient for real-time usage: They cannot provide means for real-time fine-tuning and calibration during the scanning process, nor can they provide the user with important information to manually perform such fine tuning. In other words, such systems cannot employ smartphone-embedded hardware to efficiently control the process of measurement and/or obtain feedback for the operator of that smartphone in the process of spectrometric measurement. This includes, for example, inability to ensure proper positioning in relation to the examined target object, inability to ensure proper illumination of the target, inability to allow motion compensation, and/or the like.

As shown in FIG. 3B, apparatus 10 of the present invention may be releasably attachable, via one or more affixing elements 22 such as, for example, a hook, and/or a latch, to a computing device 20 such as, for example, a smartphone.

As shown in FIG. 2, apparatus 10 may include a radiation source 120, such as, for example, a laser source, and/or a Light Emitting Diode (LED) source, configured to produce radiation of a first wavelength, e.g., in the visible spectrum and/or Near Infrared (NIR) spectrum, in a radiation direction marked by arrow DIR2A in FIG. 3B. Apparatus 10 may also include an optical system 130, configured to illuminate, and/or radiate a target object 40 with the radiation of a first wavelength, in an illumination direction marked by arrow DIR2B in FIG. 3B.

Apparatus 10 may be configured to perform spectrometric measurement 140SI of a target object 40 based on backscattering (e.g., 180 degree) geometry. In other words, optical system 130 may be configured to receive back-scattered Raman radiation from illumination direction DIR2B, e.g., in substantially 180 degrees from direction DIR2B, as depicted by arrow DIR2C. The backscattered Raman radiation DIR2C may include one or more second wavelengths, which may be indicative of substances of interest in target object 40. Apparatus 10 may analyze the radiation of DIR2C, back-scattered from direction DIR2B, to determine a spectrum of radiation reflected from target object 40, thereby identifying existence of the substances of interest in target object 40.

As elaborated herein, one advantage of the backscattering (or “180 degree”) geometry of system 100 as shown in FIG. 3B (e.g., substantially 180 degrees between DIR2B and DIR2C), is that it may allow apparatus 10 to gather information from the smartphone's 20 built-in camera 240 and/or laser autofocus sensor 250, which operate in the same direction as illumination direction DIR2B. The term operation direction may be used herein to indicate a direction for which components of the built-in camera 240 and/or laser autofocus sensor 250 are configured to receive incident radiation. For example, for camera 240, the direction of operation may substantially align with the middle of the camera's field of view.

This information may allow apparatus 10 and/or a user thereof to scan the samples object 40 efficiently. For example, apparatus 10 may collaborate with at least one processor 210 of computing device 20 (e.g., smartphone), to use using an autofocus sensor 250 that may be embedded in smartphone 20, thereby obtaining information regarding the apparatus' 10 distance from sample 40.

Additionally, or alternatively, apparatus 10 may collaborate with computing device 20 to use a camera 240 embedded in smartphone 20, and show via an output device (e.g., element 8 of FIG. 1, such as, for example, a screen of smartphone 20) an image of sample 40.

Another advantage of the backscattering 180-degree geometry is that it may enable quantification of material in target object 40. For example, target object 40 may be a glass containing alcoholic beverage. Apparatus 10 may collaborate with smartphone 10 to operate a camera 240 of computing device 20 (e.g., smartphone), to obtain an image of the beverage, and calculate the volume of the glass. Apparatus 10 may apply a Raman spectrometric measurement 140SI, to ascertain a concentration of alcohol in the drink 40, thereby calculating an absolute quantity of alcohol in the glass.

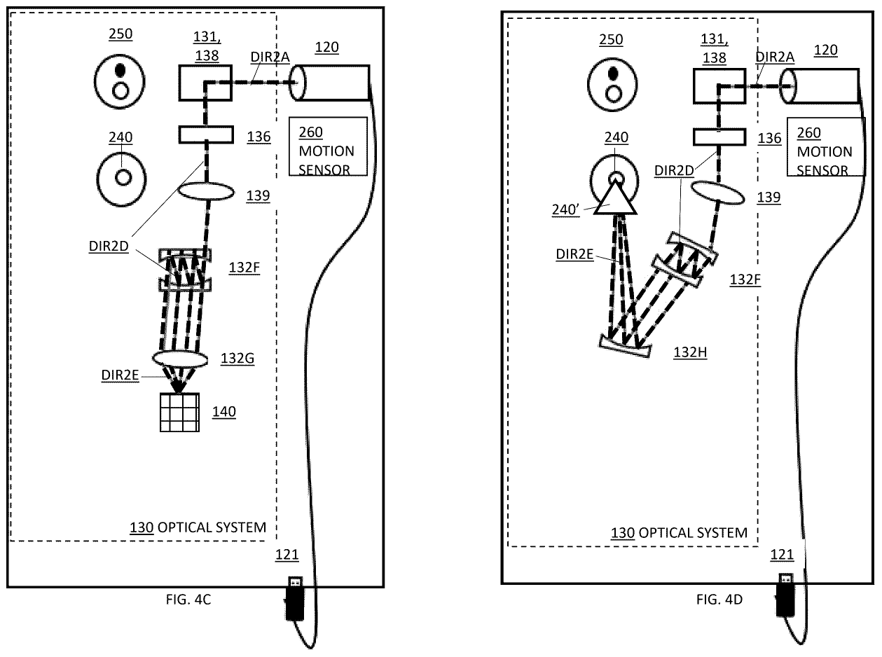

As elaborated herein (e.g., in relation to FIGS. 4A-4D), an optical system 130 that is attached to computing device 20 (e.g., smartphone), and facilitates backscattering 180 degree geometry requires a unique design of an optical path. This optical path is denoted in FIGS. 4A-4D as legs, or directions DIR2D and DIR2E.

Optical path DIR2D/DIR2E and direction DIR2A may be substantially parallel to a plane of computing device 20 (e.g., smartphone). In other words, optical path DIR2D/DIR2E and direction DIR2A may define a plane that is co-planar with apparatus 10, limited by the edges of smartphone 20, and substantially orthogonal to directions DIR2B and DIR2C (e.g., to, and from target object 40).

As elaborated herein (e.g., in relation to FIGS. 4A-4D), optical system 130 may be configured to direct the back-scattered Raman radiation DIR2C, via optical path DIR2D to a detector device, configured to measure spectral intensity of the back-scattered radiation in at least one wavelength.

For example, the detector device may be a dedicated detector 140 such as, for example, a visible light or NIR detector 140, that may be included in apparatus 10. This measured spectral intensity value is denoted herein as 140SI.

Additionally, or alternatively, the detector device may be a detector 240 of attached computing device 20 (e.g., smartphone), such as, for example, a visible light spectrum camera 240. In such embodiments, affixing element 22 may be configured to releasably attach apparatus 10 to a back side of the smartphone device 20, such that the direction of radiation (DIR2B/DIR2C) substantially aligns with a visual axis of camera 240.

As elaborated herein, system 100 may employ at least one processor (e.g., processor 2 of FIG. 1). For example, processor 2 may be a dedicated processor 110, that may be included in apparatus 10. Additionally, or alternatively, processor 2 may be a processor 210 of attached computing device 20 (e.g., smartphone). Additionally, or alternatively, processor 2 may be at least one processor included in application server 30.

According to some embodiments, processor 2 (e.g., 110, 210) may identify presence of a chemical substance of interest at the target object 40 based on the intensity of back-scattered Raman radiation DIR2C 140SI/240SI, as measured by detector device 140/240 respectively.

For example, processor 2 may obtain (e.g., from application server 30) an expected value of back-scattered radiation intensity, indicative of a substance of interest, and compare the measured spectral intensity 140SI/240SI of the back-scattered Raman radiation DIR2C from target object 40, in at least one wavelength, to determine presence of the substance of interest in target object 40.

Reference is also made to FIGS. 4A-4D which are schematic diagrams depicting different configurations of an optical system 130 of an apparatus 10 for identifying presence of a substance of interest, according to some embodiments.

As shown in FIGS. 4A-4D, optical system 130 of apparatus 10 may include, or may be associated with radiation source 120, configured to produce radiation (e.g., laser radiation) in direction DIR2A. Radiation source 120 may be powered by small, replaceable batteries. Additionally, or alternatively, radiation source 120 may be powered and/or recharged by a battery of computing device 20 (e.g., smartphone), e.g., via an appropriate cable and connector 121, such as, for example, a USB-C type connector and 121 and cable. Additionally, or alternatively, radiation source 120 may be powered by a battery of smartphone 20 via electromagnetic induction, e.g., as known in the art. e.g., by a charger (e.g., charger 60 of FIG. 4A), compliant with a wireless charging standard (e.g., the currently available Qi standard).

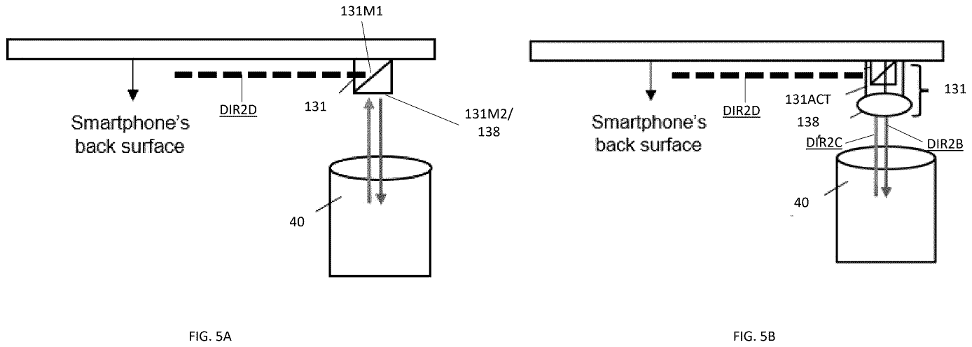

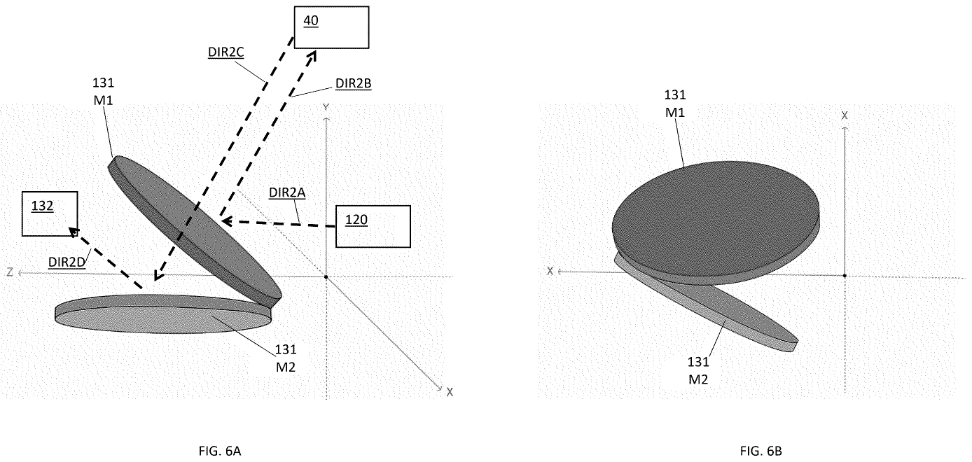

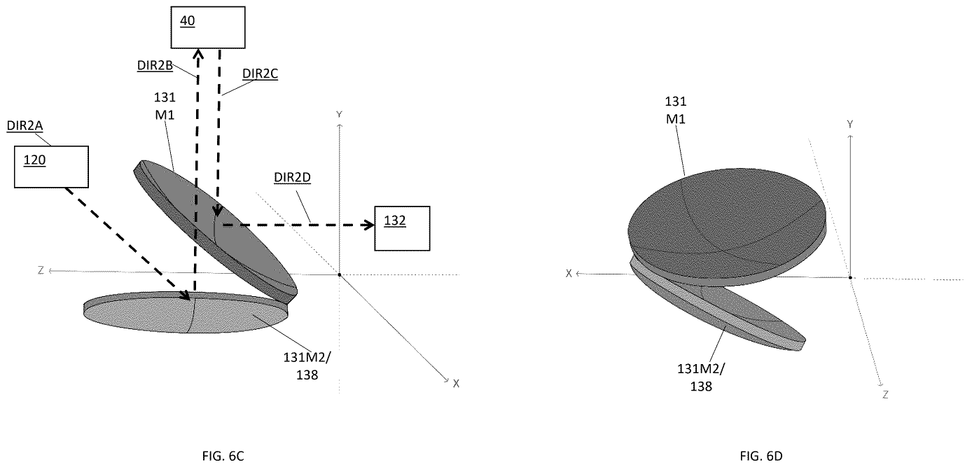

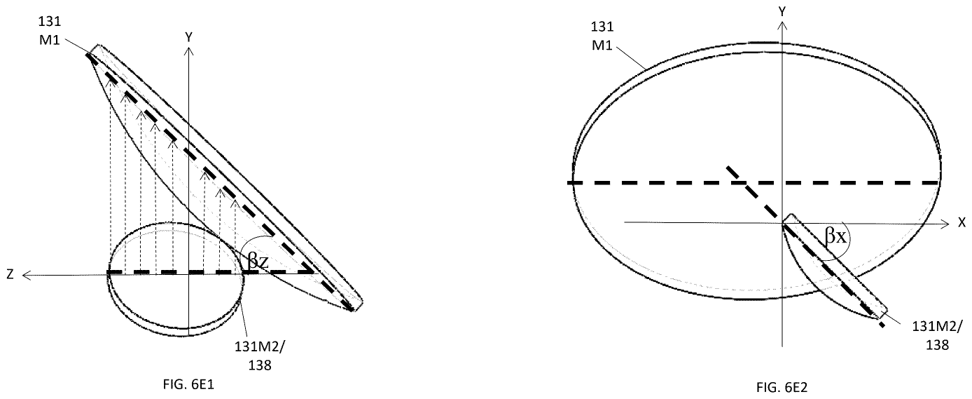

As shown in FIGS. 4A-4D, optical system 130 of apparatus 10 may include a separator component 131, which may include one or more sub-elements denoted herein as 131M1, 131M2, 131ACT, and (in some embodiments) 138. Examples of composition of the separator component 131 are brought herein, e.g., in relation to FIGS. 5A, 5B and 6A-6D.

Additionally, or alternatively, optical system 130 of apparatus 10 may include a first focusing element 138, adapted to focus the emitted radiation (e.g., in direction DIR2B) upon a surface of target object 40.

In some embodiments, focusing element 138 may be independent of separator component 131. Additionally, or alternatively, as shown for example in FIG. 4A, focusing element 138 may be integrated into separator component 131, as elaborated herein e.g., in relation to FIGS. 5A, 5B, 6C, and 6D.

Additionally, or alternatively, optical system 130 of apparatus 10 may include at least one filtering element 132. The at least one filtering element 132 may, in turn, include one or more sub-elements denoted herein as elements 132A-132E.

For example, filtering element 132 may be, or may include a Fabry-Perot resonator configuration, a Czerny-Turner spectrometer configuration, a Fastie-Ebert configuration, and/or the like. For example, as known in the art, such configurations (e.g., Fabry-Perot resonator) may be adapted to filter, pass, or select predetermined bands of scattered radiation, to be analyzed by an appropriate detector (e.g., 140/240). Elements 132A-132E may be or may include sub-parts of these configurations (e.g., of a Fabry-Perot resonator), specifically configured to enable such filtration, as elaborated in relation to each of FIGS. 4A-4D. Note that some annotations have been omitted in FIGS. 4A-4D, for the purpose of clarity.

As shown by the dashed line in each of FIGS. 4A-4D, separator component 131 of optical system 130 may be configured to reflect the radiation of radiation source (e.g., laser source) 120 to the direction DIR2B of target object 40. Separator component 131 may reflect the back-scattered radiation from the direction DIR2C of target object 40 to filtering element 132, in direction DIR2D.

Filtering element 132 may be configured to filter the back-scattered radiation from direction DIR2D, so as to obtain back-scattered radiation at a predefined band, and direct the filtered, back-scattered radiation, in direction DIR2E to a detector device 140/240.

Additionally, or alternatively, detector device 140/240 may be coupled with a corresponding optical element 140′/240′, adapted to insert the radiation from direction DIR2E into detector device 140/240 (e.g., into the detectors field of view). For example, optical element 240′ may be a mirror, adapted to change the direction of light in 90 deg, so that it may fall onto the smartphone's built-in camera 240, in the camera's direction of operation, or field of view.

As shown in the examples of FIGS. 4A and 4B, filtering element 132 may be, or may include a Czerny-Turner spectrometer configuration. For example, as known in the art, the Czerny-Turner spectrometer may include an entrance slit 132A, a collimating mirror 132B, a reflection diffraction grating module 132C, a Focusing mirror 132D, and an optional Exit slit 132E. Locations and properties of elements 132A-132E may be set, for example, as known in the art, to obtain a desired band of filtered, back-scattered radiation, in direction DIR2E.

In the example of FIG. 4B, apparatus 10 may utilize a built-in detector 240, e.g., a visible-light camera of computing device 20 (e.g., smartphone). In such embodiments, filtering element 132 (in this example, a Czerny-Turner spectrometer configuration) may be configured to fit the passband of filtered, back-scattered radiation (DIR2E) to an appropriate visible spectrum (typically 532 nm or 633 nm), so as to match the properties of camera 240.

Additionally, or alternatively, as depicted in the example of FIG. 4A, optical system 130 may include a dedicated detector 140. In such embodiments, filtering element 132 may fit the passband of filtered, back-scattered radiation (DIR2E) to a wide range of wavelengths, including for example parts of the Ultraviolet (UV) and Infrared (IR) spectrum, according to the properties of dedicated detector 140.

It may be appreciated that any combination of detectors, e.g., a plurality of dedicated detectors 140, a dedicated detector 140 and a built-in detector 240, etc. may also be possible with appropriate modifications.

As shown in the examples of FIGS. 4C and 4D, filtering element 132 may be, or may include a Fabry-Perot spectrometer configuration. For example, as known in the art, the Fabry-Perot spectrometer may include a Fabry-Perot resonator 132F and a focusing element such as, for example, a lens (element 132G of FIG. 4C) or a focusing mirror (element 132H of FIG. 4D). Locations and properties of elements 132F-132H may be set, e.g., as known in the art to, obtain a desired band of filtered, back-scattered radiation, in direction DIR2E.

In a similar manner to that described herein (e.g., in relation to FIGS. 4A and 4B), system 100 may utilize one or more detectors 140/240. The Fabry-Perot resonator 132F may be configured to produce the passband of filtered, back-scattered radiation (DIR2E) in an appropriate spectrum, so as to fit the specification of the relevant detectors 140/240. For example, as depicted in FIG. 4C, apparatus 10 may collaborate with a built-in detector (e.g., camera) 240 of computing device 20 (e.g., smartphone). In such embodiments, the Fabry-Perot resonator 132F may be configured to produce a passband of radiation DIR2E, to match a predetermined band of visible light, to which camera 240 is sensitive. In the example of FIG. 4D, apparatus 10 may include a dedicated detector 140, and the Fabry-Perot resonator 132F may be configured to produce a passband of radiation DIR2E, to match the properties of sensitivity of dedicated detector 140.

Additionally, or alternatively, optical system 130 of apparatus 10 may include a second spectral filter 136 element, adapted to block the Rayleigh scattering from radiation DIR2E and only transmit radiation in wavelength pertaining to Raman scattering toward filter 132. For example, spectral filter element 136 may be a long-pass filter, cut-on above the excitation source wavelength, or a band-stop filter such as, for example, a notch filter. In another example, spectral filter element 136 may be a short-pass filter, configured to extract the anti-stokes region, to eliminate fluorescence of target 40.

Additionally, or alternatively, optical system 130 of apparatus 10 may include a second focusing element 139, adapted to focus emitted radiation from separator component 131 (e.g., in direction DIR2D) onto an entrance pupil (e.g., 132A) of filter element 132 (e.g., Czerny-Turner spectrometer).

As shown in FIGS. 4A-4D, the optical path of optical system 130, e.g., denoted by dashed lines DIR2D and DIR2E, may be restricted or confined to edges or dimensions of smartphone device 20. Additionally, or alternatively, a plane defined by this optical path (e.g., dashed lines DIR2D and DIR2E) may be substantially parallel to the back side of smartphone device 20.

In other words, location of elements 120, 131, 132, 136, 138, 139 and 140 of optical system 130 may be arranged such that the optical path defines a plane that is substantially parallel to the smartphone's 20 back panel, and may be confined by a perimeter of the smartphone's 20 back panel.

It may be appreciated that such configuration may allow apparatus 10 to compactly fit onto a user's mobile device 20, and collaborate with mobile device 20 to provide repeatable, accurate measurement results.

Reference is also made to FIGS. 5A and 5B which are schematic diagrams depicting configurations of a separator component 131 which may be included in an apparatus 10 for identifying presence of a substance of interest, according to some embodiments.

Reference is also made to FIGS. 6A and 6B which are isometric views depicting an example of implementation of a separator component 131, which may be included in an apparatus 10 for identifying presence of a substance of interest, according to some embodiments.

For example, as known in the art, a dichroic mirror, also known as a dual-band mirror, dual-wavelength mirror, or dichroic reflector, is a specialized type of mirror which may have different optical properties at two different wavelengths. For example, a dichroic mirror may be reflective below a cut-on wavelength, and transmissive at longer wavelengths.

As shown in the examples of FIGS. 5A, 5B and 6A-6D, separator component 131 may include a pair of elements such as, for example, dichroic mirrors, denoted 131M1 and 131M2. Separator component 131 may include a pair of elements such as, for example, mirrors or lenses, denoted 131M1 and 131M2. At least one of elements 131M1 and 131M2 may be a dichroic mirror configured to reflect the radiation source 120 wavelength, and transmit radiation above the radiation source 120 wavelength. Additionally, or alternatively, at least one of elements 131M1 and 131M2 may be a dichroic mirror configured to transmit the radiation source 120 wavelength, and reflect radiation above the radiation source 120 wavelength.

For example, as shown in FIG. 6A, separator component 131 may include a pair of elements such as, for example, mirrors or lenses, denoted 131M1 and 131M2. At least one of elements 131M1 and 131M2 (e.g., 131M1) may be a dichroic mirror (e.g., a long pass dichroic mirror) configured to reflect the radiation source 120 wavelength (e.g., from direction DIR2A to target object 40 in direction DIR2B), and transmit radiation above the radiation source 120 wavelength (e.g., from direction DIR2C towards element 131M2).

In other words, as depicted in the implementation example of FIG. 6A, element 131M1 may be a first, long pass dichroic mirror, configured to receive radiation of the first wavelength from radiation source 120 in direction DIR2A, and reflect the radiation of the first wavelength in direction DIR2B, to the target object 40. Long pass dichroic mirror element 131M1 may transmit the back-scattered Raman radiation, characterized by one or more second wavelengths, received in direction DIR2C, to the second element 131M2 which may be a mirror. Mirror element 131M2 may be configured to receive the back-scattered radiation from the first mirror 131M1 from direction DIR2C, and reflect it towards filtering element 132 in direction DIR2D.

Reference is also made to FIGS. 6C and 6D which are isometric views depicting another example of implementation of a separator component 131, which may be included in an apparatus 10 for identifying presence of a substance of interest, according to some embodiments of the invention.

As depicted in the implementation example of FIG. 6C, at least one of elements 131M1 and 131M2 (e.g., 131M1) may be a dichroic mirror (e.g., a short pass dichroic mirror) configured to transmit the radiation source 120 wavelength (e.g., received from element 131M2), and reflect radiation above the radiation source 120 wavelength (e.g., from target object 40 in direction DIR2C to filtering element 132 in direction DIRDB).

In other words, element 131M2 may be a mirror, adapted to receive radiation from radiation source 120 (direction DIR2A), reflect and focus this radiation over a surface of sample object 40 in direction DIR2B. Element 131M1 may be a short pass dichroic mirror, that may pass radiation from radiation source 120 (direction DIR2B), in the radiation source 120 wavelength toward sample object 40, and reflect Raman/stokes radiation received from sample 40 in direction DIR2C towards filtering element 132 (direction DIR2D).

Reference is also made to FIGS. 6E1 and 6E2 which are diagrams of an example of a separator component 131 that may be included in an apparatus for identifying presence of a substance of interest, according to some embodiments of the invention. Separator component 131 is depicted in a 3D space represented by X, Y, and Z axes, according to some embodiments. In the example of FIGS. 6E1 and 6E2, the Y axis direction may converge with direction DIR2B/DIR2C. FIG. 6E1 shows separator component 131 projected on the YZ plane, and FIG. 6E2 shows separator component 131 projected on the XY plane.

As shown in FIGS. 6E1 and 6E2, element 131M may be positioned at a substantially 90 degrees angle in relation to rotation around the Y axis (direction DIR2B/DIR2C). In other words, the location of element 131M2 may coincide with that of element 131M1 following a 90 degree around the Y axis (direction DIR2B/DIR2C).

As shown by the thick, dashed lines in FIG. 6E1, a main diameter of a projection of element 131M1 on the YZ plane may be positioned at a substantially 45 degrees angle βz in relation to a main diameter of a projection of element 131M2 on the YZ plane.

In other words, when the Y axis converges with direction DIR2B/DIR2C (toward target 40), element (e.g., dichroic mirror) 131M1 may be positioned in angle βz (e.g., a 45-degree angle) in relation to the XZ plane.

Additionally, or alternatively, and as shown by the thick, dashed lines in FIG. 6E2, a main diameter of a projection of element 131M1 on the XY plane may be positioned at a substantially 45 degrees angle βx in relation to a main diameter of a projection of element 131M2 on the XY plane.

In other words, when the Y axis converges with direction DIR2B/DIR2C (toward target 40), element (e.g., mirror) 131M2 may be positioned in angle βx (e.g., a 45-degree angle) in relation to the XZ plane.

As shown in FIG. 6E1, element 131M2 (e.g., bottom mirror of FIG. 6C) may receive a small diameter beam (e.g., a laser beam) from radiation source 120 (e.g., laser) to focus upon sample object 40. Element 131M1 (e.g., top mirror of FIG. 6C) may be configured to collect radiation (dashed arrows) from element 131M2. Therefore, a diameter of element 131M1 may be larger than that of a corresponding diameter of element 131M2, such that a projection of this radiation will be fully collected by element 131M1 (e.g., without having the beam in direction DIR2B exceeding the 3D shape of the element 131M1).

For example, as shown in FIG. 6E1, where βz is a 45-degree angle, a dimension (e.g., diameter) of element 131M2 may be smaller than a respective dimension (e.g., diameter) of element 131M1 by at least a factor of \sqrt{2}.

In another example, element 131M2 may be positioned directly below the middle of element 131M1, and may share the same optical axis in the Y direction. A dimension (e.g., diameter) of element 131M2 may be smaller than a respective dimension (e.g., diameter) of element 131M1, as shown in Equation Eq. 1, below:

$$d = \frac{2 - \sqrt{2}}{2} D \qquad Eq . 1$$

where d is the diameter of the smaller element 131M2, and D is the diameter of the bigger element 131M1.

As shown in FIGS. 6E1 and 6E2, element 131M2 (e.g., bottom mirror of FIG. 6C) may be turned in an angle βx in relation to element 131M1, so as to allow reception of radiation from radiation source 120 (e.g., from direction DIR2A), and facilitate a flat optical path (DIR2A-DIR2E) which defines a plane that is substantially parallel to the smartphone's 20 back panel.

Additionally, or alternatively, at least one dichroic mirror (131M1/131M2) may have a concave geometry, thereby applicable as a focusing element. As elaborated herein, optical system 130 of apparatus 10 may include a focusing element 138, which may be positioned along a path of the radiation DIR2B/DIR2C, in the direction of target object 40.

According to some embodiments, shown in FIGS. 5A, 6C and 6D), at least one dichroic mirror 131M1/131M2 may have a concave geometry, thereby implementing focusing element 138. Additionally, or alternatively, as depicted in FIG. 5B, focusing element 138 may be implemented separately from separator component 131, e.g., as a lens located beyond separator component 131 in the direction DIR2B (e.g., toward target object 40).

According to some embodiments, separator component 131 may be associated with one or more electrical actuators 131ACT, configured to controllably move focusing element 138 along direction DIR2B. In such embodiments, at least one processor (e.g., 110, 210) of system 100 may be configured to operate a laser autofocus component 250 of the smartphone device, and receive therefrom an estimation of distance to a surface of target object 40. Based on the estimation of distance, the at least one processor 110/210 may control the one or more actuators 131ACT to adjust the position of focusing element 138, to focus the radiation of source 120 (e.g., in direction DIR2B) on the surface of the target object.

For example, focusing element 138 may be at least one of first mirror 131M1 and second mirror 131M2, which may be concave, and may therefore be operable as a focusing element. In such embodiments, the at least one processor 110/210 may receive, from laser autofocus component 250 of smartphone device 20 an estimation of distance to a surface of target object 40. Based on the estimation of distance, the at least one processor 110/210 may control at least one actuator 131ACT to adjust a position of the focusing element 138 (e.g., at least one of first mirror 131M1 and second mirror 131M2) along the path of the radiation in direction DIR2B, thereby focusing the radiation on the surface of target object 40.

In another example, as depicted in the example of FIG. 5B, the focusing element 138 may be a lens. In such embodiments, the at least one processor 110/210 may be further configured to receive the estimation of distance to a surface of the target object from laser autofocus component 25, and control the at least one actuator 131ACT to adjust a position of the lens 138 along the path (e.g., direction DIR2B) of radiation, thereby focusing the radiation on the surface of the target object 40.

Additionally, or alternatively, the at least one processor 110/210 may control a camera 240 of computing device 20 (e.g., smartphone), to acquire one or more (e.g., a series of) images of a surface of target object 40. The at least one processor 110/210 may analyze the acquired images, e.g., to produce frequency-space components indicative of a level of focus of the acquired images, e.g., as known in the art. The at least one processor 110/210 may subsequently control the one or more actuators 131ACT to adjust the position of focusing element 138, so as to focus the radiation of source 120 (e.g., in direction DIR2B) upon the depicted surface of the target object.

Additionally, or alternatively, the at least one processor 110/210 may receive, from laser autofocus component 250 an estimation of distance to a surface of target object 40, operate camera 240 to obtain at least one image of object 40, and based on the at least one image and estimated distance, calculate an optimal position of apparatus 10. The term “optimal” may be used in this context to refer to a relative position of apparatus 10 (and attached computing device 20 (e.g., smartphone)) for at least one of (i) focusing the radiation on the surface of the depicted target object, and (ii) centralizing, or depicting a sufficient portion of object 40 in the acquired image. The at least one processor 110/210 may subsequently display, e.g., on a screen of smartphone 20, at least one instruction for locating the apparatus 10 in the optimal position.

According to some embodiments, the at least one processor 110/210 may receive, from a motion sensor 260 (e.g., an accelerometer, a gyroscope, etc.) a motion data element 260′, representing motion of apparatus 10 (and attached device 20). In some embodiments, motion sensor 260 may be included in apparatus 10. Additionally, or alternatively, motion sensor 260 may be included or integrated in computing device 20 (e.g., smartphone). Based on motion data element 260′, the at least one processor 110/210 may control the one or more actuators 131ACT to adjust the position of the focusing element 138 (e.g., 131M2), thereby maintaining focus of the radiation of radiation source 120 on a surface of the target object 40.

Additionally, or alternatively, detector 140 may be, or may include (i) a line sensor, which includes a linear, or elongated array of radiation sensors, and (ii) a cylindrical lens, adapted to reshape the filtered, back-scattered radiation (DIR2E), so as to align with the linear array of radiation sensors. It may be appreciated that in such embodiments, each pixel of the detector 140 may collect a quantity of photons that may be equivalent to an entire column in an equivalent matrix sensor. Therefore, imaging requirements (e.g., Signal to Noise Ratio (SNR), dynamic range, absolute sensitivity threshold, and/or the like) in a line sensor based detector 140 may be alleviated, in relation to an equivalent matrix sensor. The term “equivalent” referring in this context to a detector which may cover a substantially equal area of target object 40.

Such alleviation of imaging requirements may increase simplicity, and allow reduction of cost of detector 140. Additionally, the data collected from a line-sensor based detector 140 may increase throughput of reading and analyzing data from detector 140 in comparison to an equivalent matrix sensor.

Additionally, or alternatively, detector 140 may be a matrix sensor, in which every column may be summed digitally, to emulate a line-sensor having improved signal to noise ratio (SNR).

Additionally, or alternatively, embodiments of the invention may sum each column of a matrix sensor of camera 240 of smartphone 20, such that camera 240 may act as a line-sensor, to increase data compactness and computational throughput.

As shown in FIG. 2, at least one processor (e.g., processor 110 of apparatus 10 and/or processor 210 of smartphone 20) of system 100 may communicate via communication network 50 (e.g., a cellular network) with application server 30, to obtain reference information, indicative of a substance of interest. The at least one processor 110/210 may analyze the measured spectral intensity 140SI/240SI of the filtered, backscattered Raman radiation (DIR2E) to identify presence of the substance of interest.

For example, target object 40 may be a glass containing a drink, and the substance of interest may be a suspected drug that may have been introduced into that drink. In such embodiments, the at least one processor 110/210 may obtain, from an online, or cloud server 30, a first reference spectrum data element 320, representing an expected spectral intensity of back-scattered radiation, indicative of presence of the chemical substance (e.g., the drug) in liquid.

Processor 110/210 may subsequently identify presence of the chemical substance of interest based on (i) the first, reference spectrum data element 320, and (ii) the measured spectral intensity 140SI/240SI of the filtered, backscattered Raman radiation.

In other words, if a measured spectral intensity 140SI/240SI of the backscattered Raman radiation (DIR2C/DIR2E) at one or more wavelength values corresponds to, or matches a value of the reference spectrum data element 320 at a corresponding wavelength, then processor 110/210 may determine presence of the substance of interest (e.g., chemical or drug) within target object 40 (e.g., the drink).

Additionally, or alternatively, processor 110/210 may obtain, from online or cloud server 30 a second reference spectrum data element 320, representing expected intensity of back-scattered radiation, characteristic of target object 40 (e.g., the drink). In such embodiments, processor 110/210 may identify presence of the chemical substance of interest in target object 40 (e.g., the drink), based on (i) the second reference spectrum data element 320, and (ii) the measured spectral intensity 140SI/240SI of backscattered Raman radiation DIR2C/DIR2E of the at least one wavelength.

In other words, if a measured spectral intensity 140SI/240SI of the backscattered Raman radiation (DIR2C/DIR2E) at one or more wavelength values does not correspond to, or does not match a value of the reference spectrum data element 320 at a corresponding wavelength, then processor 110/210 may determine an anomaly (e.g., presence of a suspected substance of interest) within target object 40 (e.g., the drink).

Additionally, or alternatively, processor 110/210 may obtain, e.g., via input device 7 of FIG. 1, such as, for example, a touchscreen of computing device 20 (e.g., smartphone), one or more target property data elements 220A.

Pertaining to the example of a target object 40 that is a drink, target property data elements 220A may be a drink property data element 220A, which may include for example an image of the drink (e.g., obtained via camera 240 of smartphone 20), a type of the drink (e.g., “Beer”, “Whiskey”, “Soda”, and/or the like), a brand of the drink (e.g., “Heineken”, “Johnny Walker”, “Coca-Cola”, and/or the like, respectively) a place of purchase of the drink (e.g., geolocation of a Pub, a supermarket, a restaurant, a stall, and/or the like).

Processor 110/210 may transmit, to online or cloud server 30, at least one of: (i) the one or more target object (e.g., drink) property data elements, and (ii) the measured spectral intensity 140SI/240SI (e.g., intensity of Raman backscattered Raman radiation DIR2C/DIR2E) in at least one wavelength). Processor 110/210 may subsequently obtain the reference spectrum data element, representing the relevant drink, from online or cloud server 30 in response to the transmission.

In other words, server 30 may accumulate (e.g., over time) information pertaining to specific drinks (e.g., having unique combinations of types, brands and locations of purchase) with corresponding measured spectral intensity data spectral intensity 140SI/240SI, from a plurality of apparatuses 10, as reference spectrum data elements 320. Server 30 may maintain a database (e.g., database 6 of FIG. 1) that includes the accumulated reference spectrum data elements 320 (e.g., combining measured spectral intensity values 140SI/240SI and corresponding target property values 220P).

Processor 110/210 may query database 6 of server 30 for a reference spectrum data element 320 that corresponds to specific target property values 220P, and server 30 may provide a relevant reference spectrum data element 320 to processor 110/210 in response to this query.

Additionally, or alternatively, machine learning (ML) model 310 of server 30 may be trained to predict a probability of existence of a substance of interest (e.g., a drug) within a target object 40 (e.g., a drink), as elaborated herein.

For example, ML model 310 may be a classification model, for example, pretrained by a supervised training scheme, based on an annotated training dataset, for example, as known in the art. The training dataset may include a plurality of training examples, each including a target (e.g., drink) property value 220P, and a corresponding 140SI measured spectral intensity values 140SI/240SI. Additionally, the plurality of training examples may be labeled, or annotated as pertaining to samples of target objects 40 (e.g., drinks) that contain, or do not contain specific substances of interest. ML model 310 may be trained based on the data examples in the training dataset, while utilizing the plurality of labels or annotations as supervisory information.

In some embodiments, unsupervised learning may be employed. In some embodiments, rule-based algorithms may be employed. In some examples, an (e.g., regression-based) classification model may be employed for determining whether a target contains one or more substances of interest and/or for determining a percentage and/or any other measure indicative of the amount of a substance contained in a target. In some embodiments, the output relating to the substance in the target may be provided in various manners including, for example, as a categorical output (e.g., nominal and/or ordinal); and/or a numerical output (e.g., discrete, continuous, various ranges of values). A regression-based model may relate to linear regression, or non-linear regression. In some embodiments, a regression-based model employed may include a multi-variate regression model, that is generated based on, for example, three or more parameter values relating to a target object.

One of more types of target objects may have known spectrometric characteristic. For example, whisky, champagne, wine, beer, may each exhibit known or standard spectrometric characteristics relating to, for example, their wavenumbers and corresponding intensity (e.g., slope and offset). Spectrometric measurements (e.g., Raman readings) of an observed target object may be adjusted, (re-) calibrated and/or otherwise corrected with reference to the known characteristic baseline values of the standard target object. For example, the slope and/or offset relative to a baseline intensity values obtained for an observed target object may be adjusted according to the values of the respective standard target object. Based on the adjusted, an anomaly relating to the target object may then be determined (e.g., by comparing observed intensity peaks against expected intensity peaks).

Accordingly, in some embodiments, spectrometer output values obtained in the examination of a target object having known characteristics relating to an observed target analyte (e.g., alcohol level), may be adjusted according to the reference base line values. For example, the system may store data descriptive of different types of alcoholic beverages along with data descriptive of known baseline values of these different types of alcoholic beverages. The system may be provided (e.g., by the user) with information (e.g., meta-information) about the type of beverage to be analyzed or being analyzed. Spectral baseline information of the observed target object may be compared against reference spectral baseline information stored in the system for the same type of target object (e.g., wine). If the comparison yields that the difference in the baseline information exceeds a certain threshold, the baseline level values for the observed target object may be adjusted accordingly. This way, the system may correct for changes in operating values of the measurement equipment employed, which may occur due to elevated equipment temperature caused by performing repeated measurements with the same equipment. Based on the adjusted output values, the system may determine a measure (e.g., probability) relating to the presence of an expected analyte in the observed target object (e.g., alcohol concentration in beer), and derive, based on the measured level of the expected analyte, a measure (e.g., percentage) relating to the probability of the presence of an unwanted target analyte in the object.

In some embodiments, a measure, anomaly, and/or the like, determined with respect to the presence of a target analyte (e.g., desired and/or undesired substance) in an analyzed target sample, may be determined with respect to various thresholds, e.g., by taking into consideration hysteresis.

For example, an upper threshold range may be defined for determining a measure of a target analyte (e.g., unwanted substance) in a sample. Such upper threshold range may be bounded by a high upper threshold range value and a low upper threshold range value. In case a measured value of, for example, an unwanted target analyte falls within the upper threshold range, the system may employ and/or take into consideration additional characteristics relating to the target sample including, for example, alcohol concentration, employ a more sensitive or accurate model, perform one or more additional scans of the target sample, e.g., with adjusted operating parameter values (e.g., scanning characteristics) such as, for example, higher camera gain, longer camera acquisition time, using different camera rows/columns, perform pixel binning, and/or the like. This way, in some examples, the probability of false-positives with respect to the presence of undesired target analytes may be reduced. In some examples, false-positive outputs due to noise may be reduced.

In some embodiments, a lower threshold range may be defined for determining the presence of a target analyte in a sample. For example, a lower threshold range may be defined for determining a measure relating to a desired and/or undesired target analyte in a sample. Such lower threshold range may be bounded by a high lower threshold range value and a low lower threshold range value. In case a measured value of, e.g., a desired, target analyte (e.g., alcohol concentration) falls within the lower threshold range, the system may employ and/or take into consideration additional characteristics relating to the target sample including, for example, employ a more sensitive or accurate model, perform one or more additional scans of the target sample, e.g., with adjusted scanning characteristics such as, for example, higher camera gain, longer camera acquisition time, using different camera rows/columns, perform pixel binning, and/or the like, for example, to ensure that for example alcohol concentration is indeed lowered, e.g., due to the addition of an unwanted drug. This way, the probability of outputting false-negative results with respect to the presence of undesired target analytes may be reduced.

Unless otherwise specified, the terms ‘about’ and/or ‘close’ and/or ‘substantially’ with respect to a magnitude or a numerical value may imply to be within an inclusive range of −10% to +10% of the respective magnitude or value.

The terms “substantially,” “substantial,” and/or the like, refer to a considerable degree or extent. When used in conjunction with an event or circumstance, the terms can refer to instances in which the event or circumstance occurs precisely as well as instances in which the event or circumstance occurs to a close approximation, such as, for example, accounting for typical tolerance levels or variability of the embodiments described herein.

In some examples, different (e.g., regression-based) models may be employed, depending on the scenario under which a target object is analyzed. For example, for analyzing spirits, a first model may be employed, and for beer a second model may be employed, different from the first model. For example, for analyzing a first type of spirit, a first model may be employed, and for a second type of spirit a second type of model may be employed, different from the first model.

Further scenarios that may invoke different models may include purchase location of the target object, purchase time, and/or the like.

For example, a first point-of-sale location may be associated with a higher probability of containing or about to contain undesired substances, compared to a second point-of-sale location. In some examples, a first period of time in a day may be associated with a higher probability of containing or about to contain undesired substances, compared to a second period-of-time in a day. In some examples, a first weekday may be associated with a higher probability of containing or about to contain undesired substances, compared to a second weekday, different from the first weekday.

In some examples, a purchase time-location tuple may be taken into consideration when analyzing a target object. For example, a first beverage purchase time-location tuple may be associated with a higher probability of containing or about to contain undesired substances than a beverage purchase second time-location tuple, different form the first time-location tuple.

In some examples, different types of brands, labels and/or type of beverages being analyzed may be associated with different probabilities of containing and/or about to contain undesired substances. In some examples, different types of brands, labels, and/or type of beverages, optionally by further taking into consideration purchase time and/or location, may be associated with different probabilities to contain and/or about to contain undesired substances. Clearly, additional and alternative characteristics relating to the target object may have correspondingly associated probabilities.

In some examples, associated probabilities may be updated on-the-fly, e.g., based on a scanning result of a target object, and the associated scenarios/information.

During an inference stage, server 30 may identify presence of a chemical substance of interest at the target object 40 by inferring ML based model 310 on at least one of (i) the one or more target (e.g., drink) property data elements 220P and (ii) the measured spectral intensity, 140SI/240SI of at least one Raman radiation wavelength. ML model 310 may thus predict a probability of presence of the chemical substance of interest, based on the training, as explained herein. Online server 310 may subsequently transmit the predicted value, representing probability of presence of the chemical substance, to the processor 110/210 of the relevant (e.g., the originator) apparatus 10 or computing device 20 (e.g., smartphone).

According to some embodiments, server 30 may host, or serve an online application 210APP, which may be operable via smartphone 20, acting as a client computing device.

As elaborated herein, scanned data from apparatus 10 (e.g., measured Raman spectrums 140SI/240SI) may be stored on an online, or cloud database 6.

Application 210APP may prompt a user of computing device 20 (e.g., smartphone) to input which type of drink they may be having (e.g., drink property 220P). Upon scanning of the drink by apparatus 10 (as elaborated above), the measured Raman spectrum 140SI/240SI may be labeled according to the drink property 220P.

Additionally, or alternatively, during scanning of the drink by apparatus 10, application 210APP may prompt a user to employ a camera (e.g., 240) of smartphone 20, to take a photo of the drink, and may apply an image analysis algorithm to determine which type of drink it is.

In some embodiments, the drink type (e.g., drink property 220P) may be that of a one-of-a-kind drink, in a sense that it may always be the same, all over the world. Such type of drink may be a beverage of a specific brand in a specific variation, e.g., Weihenstephaner Vitus beer. For this type of drink, one non-spiked reference (e.g., where a substance of interest is not present) may be enough for the algorithm. Additional samples may be taken into consideration with some weighting method.

Additionally, or alternatively, the drink property data element 220P may include a known type of the drink (e.g., “Red wine”), but the manufacturer or brand may not be known. This type of drink may basically be similar each time, but could present greater variance between measured spectral values 140SI/240SI. For such drink property 220P, Application 210APP may take into consideration all relevant reference spectra 320 pertaining to the same type (e.g., red wine) with some weighting method.

Additionally, or alternatively, the drink property data element 220P may include, or represent in-house variations of drinks (e.g., locally fixed cocktail drinks). For such drink property data type 220P, application 210APP may take into consideration various manners by which such beverages may be mixed or diluted. Additionally, the measured spectral values 140SI/240SI may be associated with a label, identifying a specific location, such as, for example, a name, address and/or geolocation of a place of purchase (e.g., a pub).

Processor 110/210 may query cloud server 30 to find the most relevant reference spectra for this type of drink, taking into account the location and type of the scanned drink. Application 210APP may use the location data for weighing specific reference spectra, to provide a weighted average of the relevant type of drink from all locations.

Additionally, or alternatively, the drink property data element 220P may include, or represent an unknown beverage or cocktail. Such drinks may include a plurality of unknown substances. According to some embodiments, ML model 310 may identify the correct combination of substances by analyzing the measured Raman spectrum values 140SI/240SI (e.g., wavelengths and amplitudes of Raman radiation peaks), determine quantity of the various identified substances, and select the most accurate reference spectrum data element 320 for the scanned beverage.

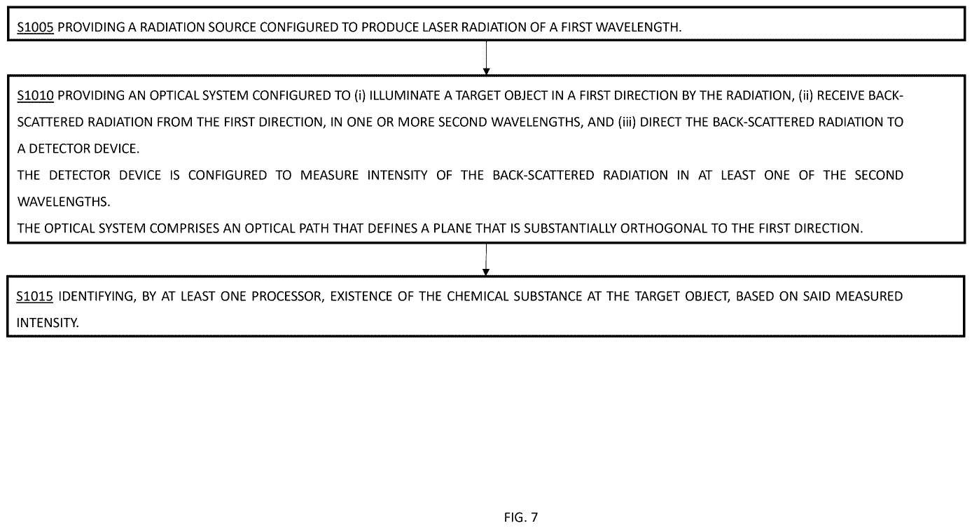

Reference now made to FIG. 7, which is a flow diagram depicting a method of identifying presence of a substance of interest in a target object 40, according to some embodiments of the invention.

As shown in step S1005, embodiments of the invention may include providing a radiation source (e.g., source 120 of FIGS. 4A-4D), configured to produce radiation of a first wavelength.

As shown in step S1010, embodiments of the invention may include providing an optical system (e.g., 130 of FIGS. 4A-4D), configured to (i) illuminate a target object 40 in a first direction (e.g., DIR2B of FIGS. 4A-4D) by the radiation; (ii) receive back-scattered radiation from the first direction (e.g., DIR2C of FIGS. 4A-4D, substantially 180-degrees from DIR2B) in one or more second wavelengths; and (iii) direct (e.g., DIR2D/DIR2E) the back-scattered radiation to a detector device (e.g., 140/240 of FIGS. 4A-4D). The detector device may be configured to measure intensity of the back-scattered radiation (DIR2C) in at least one of the second wavelengths. The optical system 130 may include an optical path (DIR2D/DIR2E and/or DIR2A) that defines a plane which is substantially orthogonal to the first direction (DIR2B/DIR2C).

As shown in step S1015, embodiments of the invention may include identifying, by at least one processor or controller (110/210), existence of the chemical substance at the target object, based on the measured intensity.

As elaborated herein, embodiments of the invention include an apparatus and a practical application for conveniently utilizing a smartphone as a reliable, cost effective and repeatable Raman spectrometer, which may allow determining existence of a substance of interest (e.g., a drug) within a target object (e.g., a drink).

It is noted that embodiments and examples disclosed herein may be applicable to alcoholic and non-alcoholic beverages.

### Additional Examples

Example 1 pertains to a system for detecting one or more target analytes in a sample or target object to be analyzed, the system comprising:
at least one processor; and
at least one memory configured to store data and software code portions executable by the at least one processor to cause performing the following steps:
determining a level of a first target analyte in the sample;
sensing, by at least one sensor, a level of a second target analyte in the sample, wherein the second target analyte is different from the first target analyte; and
determining, based on the level of the first target analyte, a probability with respect to an anomaly of the level of the second target analyte in the sample.

Example 2 includes the subject matter of example 1 and, optionally, wherein the first target analyte includes ethanol in an alcoholic beverage; and wherein the second target analyte pertains to an incapacitating substance in the alcoholic beverage.

Example 3 includes the subject matter of any one of the examples 1 to 2 and, optionally, wherein the at least one sensor includes one or more different or identical sensors.

Example 4 includes the subject matter of any one of the examples 1 to 3 and, optionally, wherein determining the level of the first target analyte and/or the second target analyte is performed by employing spectral analysis of the target sample.

Example 5 includes the subject matter of any one of the examples 1 to 4 and, optionally, wherein the determining, based on the level of the first target analyte, the probability with respect to an anomaly of the level of the second target analyte in the sample, comprises:

comparing a measured level of the first target analyte against an expected level of the first target analyte.

Example 6 includes the subject matter of example 5 and, optionally, determining an increased probability of the presence of the second target analyte, if it is determined that the measured level of the first target analyte is below the expected level of the first target analyte.

Example 7 includes the subject matter of examples 5 and/or 6 and, optionally, wherein the expected level of the first target analyte in the sample is derived through metadata relating to the target object.

Example 8 includes the subject matter of example 7 and, optionally, wherein the system is configured to acquire the information about the expected level of the first target analyte printed on a label physically attached to a receptacle containing the target object.

Example 9 includes the subject matter of any one or more of the examples 1 to 8 and, optionally, wherein the at least one sensor outputs spectral information about the target object for processing.

Example 10 includes the subject matter of example 9 and, optionally, wherein the processing of the spectral measurement output includes:

adjusting the spectral measurement output with respect to a reference baseline function of spectral measurement outputs relating to known first target analyte values in a reference target object.

Example 11 includes the subject matter of any one or more of the examples 1 to 10 and, optionally, wherein the steps further comprise:

determining, based on a measured level of the second target analyte, whether there is an anomaly or not with respect to a measured level of the second target analyte in the sample.

Example 12 includes the subject matter of any one or more of the examples 1 to 11 and, optionally, wherein the first target analyte comprises an incapacitating agent;

wherein the second target analyte comprises ethanol in an alcoholic beverage.

Example 13 includes the subject matter of any one or more of the examples 1 to 12 and, optionally, wherein the steps further comprise:
distinguishing between:
a first anomaly, which pertains to the presence of an incapacitating substance in an alcoholic beverage; and
a second anomaly, which pertains to an ethanol quantity in the alcoholic beverage.

Example 14 includes the subject matter of any one or more of the examples 1 to 13 and, optionally, wherein if a sensed measured quantity of the second target analyte is within an upper threshold range, the steps further comprise:

performing at least one additional test of the sample by employing one or more second testing parameter values which are different from those employed in a previous test.

Example 15 includes the subject matter of example 14 and, optionally, wherein the user is prompted to initiate the at least one additional test.

Example 16 includes the subject matter of any one or more of the examples 1 to 15 and, optionally, wherein the target object is a drink, and wherein the at least one processor is further configured to:
obtain, from an online server, a second reference spectrum data element, representing expected intensity of back-scattered radiation, characteristic of the drink; and
identify presence of the chemical substance of interest in the drink, based on
i) the obtained second reference spectrum data element stored in the memory, and
ii) the measured intensity of the at least one second wavelength.

Example 17 includes the subject matter of example 16 and, optionally, wherein the at least one processor is further configured to:
obtain one or more drink property data elements, selected from a list consisting of: an image of the drink, a type of the drink, a brand of the drink, and a place of purchase of the drink;
transmit, to the online server, at least one of:
(i) the one or more drink property data elements, and (ii) said measured intensity of the at least one second wavelength; and
obtain the second reference spectrum data element from the online server in response to the transmission.

Example 18 includes the subject matter of example 16 and/or example 17 and, optionally, wherein identifying presence of a chemical substance of interest at the target object comprises:
inferring, by an online server, a machine learning (ML) based model on at least one of (i) the one or more drink property data elements, and (ii) the measured intensity of the at least one second wavelength, to predict a probability of presence of the chemical substance of interest; and
transmitting, by the online server, the predicted value to the at least one processor of the apparatus.

Example 19 pertains to a system for detecting one or more target analytes in a sample or target object to be analyzed, the system comprising:
at least one processor; and
at least one memory configured to store data and software code portions executable by the at least one processor to cause performing the following steps:
sensing, by at least one sensor, a level of an undesired substance in the sample;
sensing, by the at least one sensor, a level of a desired substance in the sample; and
determining, based on the level of the first undesired substance, an expected level of the desired substance in the sample. Example 19 may be combined with any one or more of the examples 1 to 18, where applicable.

Example 20 pertains to an apparatus for detecting a chemical substance, the apparatus comprising:
a radiation source configured to produce radiation of a first wavelength;
an optical system configured to illuminate a target object in a first direction by the radiation,
receive back-scattered radiation from the first direction, in one or more second wavelengths, and
direct the back-scattered radiation to a detector device, wherein the detector device is configured to measure intensity of the back-scattered radiation in at least one of the second wavelengths; and
at least one processor configured to identify presence of a chemical substance of interest at the target object, based on said measured intensity,
wherein the optical system comprises an optical path that defines a plane that is substantially orthogonal to the first direction.

Example 21 pertains to an apparatus for detecting a chemical substance, the apparatus comprising:
a radiation source configured to produce radiation of a first wavelength;
an optical system configured to (i) illuminate a target object in a first direction by the radiation, (ii) receive back-scattered radiation from the first direction, in one or more second wavelengths, and (iii) direct the back-scattered radiation to a detector device, wherein the detector device is configured to measure intensity of the back-scattered radiation in at least one of the second wavelengths; and
at least one processor configured to identify presence of a chemical substance of interest at the target object, based on said measured intensity,
wherein the optical system comprises an optical path that defines a plane that is substantially orthogonal to the first direction.

Example 22 includes the subject matter of example 21 and, optionally, wherein the optical system comprises:
a separator component, configured to (i) reflect the radiation to the target object in the first direction, and (ii) reflect the back-scattered radiation from the first direction to a filtering element in a second direction;
the filtering element, configured to (iii) filter the back-scattered radiation, so as to obtain back-scattered radiation at a predefined band, and (iv) direct the filtered, back-scattered radiation to the detector device.

Example 23 includes the subject matter of any one or more of the examples 21 to 22 and, optionally, wherein the detector device is a camera of a smartphone device, and wherein the apparatus further comprises an affixing element, configured to releasably attach the apparatus to a back side of the smartphone device, such that the first direction substantially aligns with a visual axis of the camera.

Example 24 includes the subject matter of any one or more of the examples 21 to 23 and, optionally, wherein the optical path is restricted to edges of the smartphone device, and wherein the plane defined by the optical path that is substantially parallel to the back side of the smartphone device.

Example 25 includes the subject matter of any one or more of the examples 21 to 24 and, optionally, wherein the optical system further comprises a focusing element, positioned along a path of the radiation in the first direction, and wherein the at least one processor is further configured to:
receive, from a laser autofocus component of the smartphone device, an estimation of distance to a surface of the target object; and
based on said estimation of distance, control at least one actuator to adjust the position of the focusing element, thereby focusing the radiation on the surface of the target object.

Example 26 includes the subject matter of any one or more of the examples 21 to 25 and, optionally, wherein the at least one processor is further configured to:
receive, from a laser autofocus component of the smartphone device, an estimation of distance to a surface of the target object;
operate the camera, to obtain at least one image of the object;
based on said image and estimated distance, calculate an optimal position of the apparatus for focusing the radiation on the surface of the target object; and
display, on a screen of the smartphone, at least one instruction for locating the apparatus in said optimal position.

Example 27 includes the subject matter of any one or more of the examples 21 to 26 and, optionally, wherein the at least one processor is further configured to:
receive, from a motion sensor of the smartphone, a motion data element, representing motion of the apparatus; and
based on said motion data element, control the at least one actuator to adjust the position of the focusing element, thereby maintaining focus of the radiation on the surface of the target object.

Example 28 includes the subject matter of any one or more of the examples 21 to 27 and, optionally, wherein the separator component comprises:
a first, dichroic mirror, configured to (i) reflect the radiation of the first wavelength to the target object, and (ii) transmit the back-scattered radiation to a second mirror; and
the second mirror, configured to receive the back-scattered radiation from the first mirror and reflect it towards the filtering element in the second direction.

Example 29 includes the subject matter of any one or more of the examples 21 to 28 and, optionally, wherein the separator component comprises:
a first, dichroic mirror, configured to (i) transmit the radiation of the first wavelength to the target object in the first direction, and (ii) reflect the back-scattered radiation from the target object to the filtering element in the second direction; and
a second mirror, configured to receive the radiation of the first wavelength from the radiation source, and reflect it towards the first mirror.

Example 30 includes the subject matter of any one or more of the examples 21 to 29 and, optionally, wherein the focusing element is the first mirror, and wherein the first mirror is concave, and wherein the at least one processor is further configured to:
receive, from a laser autofocus component of the smartphone device, an estimation of distance to a surface of the target object; and
based on said estimation of distance, control at least one actuator to adjust a position of the first mirror along the path of the radiation in the first direction, thereby focusing the radiation on the surface of the target object.

Example 31 includes the subject matter of any one or more of the examples 21 to 30 and, optionally, wherein the focusing element is a lens, and wherein the at least one processor is further configured to:
receive, from a laser autofocus component of the smartphone device, an estimation of distance to a surface of the target object; and

Example 32 includes the subject matter of any one or more of the examples 21 to 31 and, optionally, based on said estimation of distance, control at least one actuator to adjust a position of the lens along the path of the radiation in the first direction, thereby focusing the radiation on the surface of the target object.

Example 33 includes the subject matter of any one or more of the examples 21 to 32 and, optionally, wherein the filtering element is selected from a list consisting of: a Fabry-Perot resonator configuration, a Czerny-Turner configuration, and a Fastie-Ebert configuration.

Example 34 includes the subject matter of any one or more of the examples 21 to 33 and, optionally, wherein the detector device comprises:
a line sensor, comprising a linear array of radiation sensors; and
a cylindrical lens, adapted to reshape the filtered, back-scattered radiation to align with the linear array of radiation sensors.

Example 35 includes the subject matter of any one or more of the examples 21 to 34 and, optionally, wherein the at least one processor is further configured to:
obtain, from an online server, a first reference spectrum data element, representing expected intensity of back-scattered radiation, indicative of presence of the chemical substance in liquid; and
identify presence of the chemical substance of interest based on (i) the first spectrum data element, and
(ii) said measured intensity of the at least one second wavelength.

Example 36 includes the subject matter of any one or more of the examples 21 to 35 and, optionally, wherein the target object is a drink, and wherein the at least one processor is further configured to:
obtain, from an online server, a second reference spectrum data element, representing expected intensity of back-scattered radiation, characteristic of said drink; and
identify presence of the chemical substance of interest in the drink, based on (i) the second reference spectrum data element, and (ii) said measured intensity of the at least one second wavelength.

Embodiments of the invention may include an apparatus for detecting a chemical substance. According to some embodiments, the apparatus may include a radiation source configured to produce radiation of a first wavelength; an optical system configured to (i) illuminate a target object in a first direction by the radiation, (ii) receive back-scattered radiation from the first direction, in one or more second wavelengths, and (iii) direct the back-scattered radiation to a detector device. The optical system may include an optical path that defines a plane that may be substantially orthogonal to the first direction.

The detector device may be configured to measure intensity of the back-scattered radiation in at least one of the second wavelengths. The apparatus may further include at least one processor, configured to identify presence of a chemical substance of interest at the target object, based on the measured intensity.

According to some embodiments, the optical system may include a separator component, configured to (i) reflect the radiation to the target object in the first direction, and (ii) reflect the back-scattered radiation from the first direction to a filtering element in a second direction; and the filtering element, configured to (iii) filter the back-scattered radiation, so as to obtain back-scattered radiation at a predefined band, and (iv) direct the filtered, back-scattered radiation to the detector device.

According to some embodiments, the detector device may be a camera of a computing device such as, for example, a smartphone, and/or tablet computer. The apparatus may further include an affixing element, configured to releasably attach the apparatus to a back side of the computing device (e.g., smartphone), such that the first direction substantially aligns with a visual axis of the camera.

According to some embodiments, the optical path may be restricted to edges of the computing device (e.g., smartphone), and the plane defined by the optical path may be substantially parallel to the back side of the computing device.

The optical system may further include a focusing element, positioned substantially along a path of the radiation in the first direction.

The at least one processor may be further configured to receive, from a laser autofocus component of the computing device (e.g., smartphone), an estimation of distance to a surface of the target object; and based on the estimation of distance, control at least one actuator to adjust the position of the focusing element, thereby focusing the radiation on the surface of the target object.

Additionally, or alternatively, the at least one processor may be further configured to receive, from a laser autofocus component of the computing device (e.g., smartphone), an estimation of distance to a surface of the target object; operate the camera, to obtain at least one image of the object; based on the image and estimated distance, calculate an optimal position of the apparatus for focusing the radiation on the surface of the target object; and display, on a screen of the computing device (e.g., smartphone), at least one instruction for locating the apparatus in the optimal position.

Additionally, or alternatively, the at least one processor may be further configured to receive, from a motion sensor of the computing device (e.g., smartphone), a motion data element, representing motion of the apparatus; and based on the motion data element, control the at least one actuator to adjust the position of the focusing element, thereby maintaining focus of the radiation on the surface of the target object.

According to some embodiments, the separator component may include a first mirror (e.g., a dichroic mirror), configured to (i) reflect the radiation of the first wavelength to the target object, and (ii) transmit the back-scattered radiation to a second mirror (e.g., a dichroic mirror); and the second mirror, configured to receive the back-scattered radiation from the first mirror and reflect it towards the filtering element in the second direction.

Additionally, or alternatively, the focusing element may be the first mirror. For example, the first mirror may be concave, and thereby operate as the focusing element. In such embodiments, the at least one processor may be further configured to receive, from a laser autofocus component of the computing device (e.g., smartphone), an estimation of distance to a surface of the target object; and based on the estimation of distance, control at least one actuator to adjust a position of the first mirror along the path of the radiation in the first direction, thereby focusing the radiation on the surface of the target object.

Additionally, or alternatively, the focusing element may be a lens. The at least one processor may be further configured to receive, from a laser autofocus component of the computing device (e.g., smartphone), an estimation of distance to a surface of the target object; and based on the estimation of distance, control at least one actuator to adjust a position of the lens along the path of the radiation in the first direction, thereby focusing the radiation on the surface of the target object.

According to some embodiments, the filtering element may be, or may include a Fabry-Perot resonator configuration, a Czerny-Turner configuration, and a Fastie-Ebert configuration.

According to some embodiments, the detector device may be, or may include a line sensor, having a linear array of radiation sensors. In such embodiments, the detector device may further include a cylindrical lens, adapted to reshape the filtered, back-scattered radiation to align with the linear array of radiation sensors.

Additionally, or alternatively, the at least one processor may be further configured to obtain, from an online server, a first reference spectrum data element, representing expected intensity of back-scattered radiation, indicative of presence of the chemical substance in liquid; and identify presence of the chemical substance of interest based on (i) the first spectrum data element, and (ii) the measured intensity of the at least one second wavelength.

For example, the target object may be a drink, and the at least one processor may be further configured to obtain, from the online server, a second reference spectrum data element, representing expected intensity of back-scattered radiation, characteristic of the drink. The at least one processor may subsequently identify presence of the chemical substance of interest in the drink, based on (i) the second reference spectrum data element, and (ii) the measured intensity of the at least one second wavelength.

Additionally, or alternatively, the at least one processor may be further configured to obtain one or more drink property data elements, such as, for example, an image of the drink, a type of the drink, a brand of the drink, a place of purchase of the drink, and/or the like. The at least one processor may then transmit (e.g., via a cellular network) to the online server, at least one of: (i) the one or more drink property data elements, and (ii) the measured intensity of the at least one second wavelength. The at least one processor may obtain the second reference spectrum data element from the online server in response to the transmission.

Additionally, or alternatively, the online server may be configured to infer a machine learning (ML) based model on at least one of (i) the one or more drink property data elements, and (ii) the measured intensity of the at least one second wavelength, to predict a probability of presence of the chemical substance of interest. The online server may then transmit the predicted value to the at least one processor of the apparatus, to identify presence of a chemical substance of interest at the target object based on the predicted probability.

Embodiments of the invention may include a method of identifying presence of a chemical substance of interest in a target object. Embodiments of the method may include providing a radiation source (e.g., 120) configured to produce radiation of a first wavelength; providing an optical system (e.g., 130) configured to (i) illuminate a target object in a first direction by the radiation, (ii) receive back-scattered radiation from the first direction, in one or more second wavelengths, and (iii) direct the back-scattered radiation to a detector device, wherein the detector device may be configured to measure intensity of the back-scattered radiation in at least one of the second wavelengths; and identifying, by at least one processor (e.g., 110/210) existence of the chemical substance at the target object, based on the measured intensity. The optical system may include an optical path (e.g., DIR2D/DIR2E) that defines a plane that may be substantially orthogonal to the first direction (e.g., DIR2B/DIR2C).

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

It is appreciated that certain features of the invention, which are, for clarity, described in the context of separate embodiments, examples, and/or the like, may also be provided in combination in a single embodiment. Conversely, various features of the invention, which are, for brevity, described in the context of a single embodiment, example and/or option, may also be provided separately or in any suitable subcombination or as suitable in any other described embodiment, examples, and/or the like of the invention.

Certain features described in the context of various embodiments are not to be considered essential features of those embodiments, unless the embodiment, example, and/or option is inoperative without those elements.

Features, structures, characteristics, stages, methods, modules, elements, entities or systems disclosed herein, which are, for clarity, described in the context of separate examples, may also be provided in combination in a single example.

Correspondingly, combination of features disclosed in different embodiments can also be considered as being disclosed in the present description.

Conversely, various features, structures, characteristics, stages, methods, modules, elements, entities or systems disclosed herein, which are, for brevity, described in the context of a single example, embodiment, and/or the like, may also be provided separately or in any suitable sub-combination.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as, for example, from 1 to 6 should be considered to have specifically disclosed subranges such as, for example, from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not to be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

Thresholds disclosed herein may be predetermined thresholds, adaptively or dynamically updated thresholds. Predetermined thresholds may be thresholds that remain static. Dynamic thresholds are forcefully changed, for example, at a certain time of day, or a certain day of the year. Adaptive thresholds are changed in response to, for example, changes in characteristics relating to an analyzing scenario (e.g., depending on the target object being analyzed, location of the analysis, beverage brand, label attached to a receptacle, type of beverage being analyzed, and/or the like).

What is claimed is:

1. A system for detecting one or more target analytes in a sample or target object to be analyzed, the system comprising:
at least one processor; and
at least one memory configured to store data and software code portions executable by the at least one processor to cause performing the following steps:
receiving metadata relating to the sample;
interrogating the sample by at least one sensor for obtaining data descriptive of the sample;
determining, based on the metadata and the obtained data descriptive of the sample, information about the presence of one or more target analytes in the sample.

2. The system of claim 1,
wherein the one or more target analytes comprise:
a first target analyte that pertains to an incapacitating substance in the sample.

3. (canceled)

4. The system of claim 1, comprising an apparatus for performing the interrogating, the apparatus comprising:
a radiation source configured to produce radiation of a first wavelength;
an optical system configured to
(i) illuminate a target object in a first direction by the radiation,
(ii) receive back-scattered radiation from the first direction, in one or more second wavelengths, and
(iii) direct the back-scattered radiation to a detector device, wherein the detector device is configured to measure intensity of the back-scattered radiation in at least one of the second wavelengths; and
at least one processor configured to identify presence of a chemical substance of interest at the target object, based on said measured intensity,
wherein the optical system comprises an optical path that defines a plane that is substantially orthogonal to the first direction.

5. The system of claim 2, further comprising:
determining, based on the level of the first target analyte, the probability with respect to an anomaly of the level of a second target analyte in the sample.

6. The system of claim 5, determining an increased probability of the presence of the second target analyte, if it is determined that the measured level of the first target analyte is below the expected level of the first target analyte.

7. The system of claim 1, wherein the metadata pertains to a purchase location of the sample, and/or information printed on a label associated with the sample.

8. The system of claim 7, configured to acquire the information about the expected level of the first target analyte printed on a label physically attached to a receptacle containing the target object.

9. (canceled)

10. (canceled)

11. The system of claim 1, wherein the steps further comprise:
determining, based on a measured level of the second target analyte, whether there is an anomaly or not with respect to a measured level of the second target analyte in the sample.

12. The system of claim 1,
wherein the first target analyte comprises an incapacitating agent;
wherein the second target analyte comprises ethanol in an alcoholic beverage.

13. The system of claim 1, wherein the steps further comprise:
distinguishing between:
a first anomaly, which pertains to the presence of an incapacitating substance in an alcoholic beverage; and
a second anomaly, which pertains to an ethanol quantity in the alcoholic beverage.

14. The system of claim 1, wherein:
if a sensed measured quantity of the second target analyte is within an upper threshold range, the steps further comprise:
performing at least one additional test of the sample by employing one or more second testing parameter values which are different from those employed in a previous test.

15. The system of claim 14, wherein the user is prompted to initiate the at least one additional test.

16. The system of claim 1, wherein the target object is a drink, and wherein the at least one processor is further configured to:
obtain, from an online server, a second reference spectrum data element, representing expected intensity of back-scattered radiation, characteristic of the drink; and
identify presence of the chemical substance of interest in the drink, based on
(i) the obtained second reference spectrum data element stored in the memory, and
(ii) the measured intensity of the at least one second wavelength.

17. The system of claim 16, wherein the at least one processor is further configured to:
obtain one or more drink property data elements, selected from a list consisting of: an image of the drink, a type of the drink, a brand of the drink, and a place of purchase of the drink;
transmit, to the online server, at least one of:
(i) the one or more drink property data elements, and
(ii) said measured intensity of the at least one second wavelength; and
obtain the second reference spectrum data element from the online server in response to the transmission.

18. The system of claim 16, wherein identifying presence of a chemical substance of interest at the target object comprises:
inferring, by an online server, a machine learning (ML) based model on at least one of the one or more drink property data elements, and
the measured intensity of the at least one second wavelength, to predict a probability of presence of the chemical substance of interest; and
transmitting, by the online server, the predicted value to the at least one processor of the apparatus.

19. A system for detecting one or more target analytes in a sample or target object to be analyzed, the system comprising:
at least one processor; and
at least one memory configured to store data and software code portions executable by the at least one processor to cause performing the following steps:
sensing, by at least one sensor, a level of an undesired substance in the sample;
sensing, by the at least one sensor, a level of a desired substance in the sample; and
determining, based on the level of the first undesired substance, an expected level of the desired substance in the sample.

20. (canceled)

21. An apparatus for detecting a chemical substance, the apparatus comprising:
a radiation source configured to produce radiation of a first wavelength;
an optical system configured to
(i) illuminate a target object in a first direction by the radiation,
(ii) receive back-scattered radiation from the first direction, in one or more second wavelengths, and (iii) direct the back-scattered radiation to a detector device, wherein the detector device is configured to measure intensity of the back-scattered radiation in at least one of the second wavelengths; and
at least one processor configured to identify presence of a chemical substance of interest at the target object, based on said measured intensity,
wherein the optical system comprises an optical path that defines a plane that is substantially orthogonal to the first direction.

22. The apparatus of claim 21, wherein the optical system comprises:
a separator component, configured to
(i) reflect the radiation to the target object in the first direction, and
(ii) reflect the back-scattered radiation from the first direction to a filtering element in a second direction;
the filtering element, configured to
(iii) filter the back-scattered radiation, so as to obtain back-scattered radiation at a predefined band, and
(iv) direct the filtered, back-scattered radiation to the detector device.

23. The apparatus of claim 21, wherein the detector device is a camera of a smartphone device, and wherein the apparatus further comprises an affixing element, configured to releasably attach the apparatus to a back side of the smartphone device, such that the first direction substantially aligns with a visual axis of the camera.

24. The apparatus of claim 21, wherein the optical path is restricted to edges of the smartphone device, and wherein the plane defined by the optical path that is substantially parallel to the back side of the smartphone device.

25. The apparatus of claim 21, wherein the optical system further comprises a focusing element, positioned along a path of the radiation in the first direction, and wherein the at least one processor is further configured to:
receive, from a laser autofocus component of the smartphone device, an estimation of distance to a surface of the target object; and
based on said estimation of distance, control at least one actuator to adjust the position of the focusing element, thereby focusing the radiation on the surface of the target object.

26. The apparatus of claim 21, wherein the at least one processor is further configured to:
receive, from a laser autofocus component of the smartphone device, an estimation of distance to a surface of the target object;
operate the camera, to obtain at least one image of the object;
based on said image and estimated distance, calculate an optimal position of the apparatus for focusing the radiation on the surface of the target object; and
display, on a screen of the smartphone, at least one instruction for locating the apparatus in said optimal position.

27. (canceled)

28. (canceled)

29. (canceled)

30. (canceled)

31. (canceled)

32. (canceled)

33. (canceled)

34. (canceled)

35. (canceled)
